SEQUENCE LISTING (123 sequences)

SEQ ID NO: 1 - DNA, 4964 bp, Mus musculus
  source 1..4964: mol_type = genomic DNA
cgcatggacctagtcgggggcgctggctcagccccgccccgcgcgcccccgtcgcagccgacgcgcgctcccgggaggcggcggcagaggcagcatccacagcatcagcagcctcagcttcatccccgggcggtctccggcggggaaggccggtgggacaaacggacagaaggcaaagtgcccgcaatggagggagcatcctttggcgcgggccgtgcgggagctgcctttgatcccgtgagctttgcgcggcggccccagaccctgttgcgggtcgtgtcctgggtgagtggtccctaagctggccccttccctgtcccccgcctctcttagcatcccttatcctccaccctcacccaccccctgcccatcgcccctcaccctcctctccgccggtccctccccccaccccccgcccgcaggttggggtgacgtcacatggctaggcgcgcccacctgcgggcgggggaagggacggcggctgttaccaatagaccttgagaggtcaccactggtctccccctgccctcaattcctcttaaggtctaatacagtccccttcctaggtcttttcatttttccggttcccacgacctggaagtaggcacagagttgcggatgtaattctcccagtatatcagggacttaattccacatggaagagggggtataggagccaaggagaatgcgcagtttcttccctttgcctgataacatccgccaggctcatttggagggagcggttcttcctagatccctcaggtctagctgtgagttaaaggagcataaggaggctccctgctgctgtagctagaaggggctcatctgggagggcttcctggaaatggcatggaagctagagacggggagtaatctatctcattagcgaatgtcttccctgtcctaggcatttaggagatgggtgctaagttctggattttggattttgtccatctgggagtcctctaatctccatgcctgtcttgaaagagggcggttcccagggtggagccccacccccaccccccaagacctcgtggactgaaggaagaacagtctctggtcctgacttgtggattgctttcaagaaacatgagcatgctgtggcgtttttcatgactacattatcaaggagtttagttgagaattgtggaatgataggagttgagatgtttcttagaaatgtcctagacgtctgcactaaaatgactgtggtttgcttctgtcctgccttggagaactagttggtgggcagagggtcctgaggttggctcaaaccttagagcctcaatgctcttgactgatattatgataactgactactcacgggaatctattctgaaatgcccactattgctggtggacattcttggacccagcattccaaactaggctccttgacaccatgtgggacactgaggagcacacacacacacaggtcctgtgtgcagaggtcaggttcgaaaagggaaaggcttgtatatttctctagacagctgtcccgtgacttcactctccacagtgcactggcccaccacaaccctggaaatcactgatgtaggtgaaggctcctgcttccacgcttccagtaaactctgggaggccataaggttctaagaccggaggagtgactgccctgagaagtacagtgtgaaccctttatactcccttataaaggggataagagtggcacctctgatatttttgatttagatcacctgtaactaagctacagtctgcagctgccatgtgggtgctgagaagtgaacacaggtcctcttaaccgcagagccatatgtcagccctgtattagagcctcaagtagccaaggctgtcttcctctgatccttcttactccacctcccgatcaattgttgagattacaggcatgcacctccttgcaaacgtaaatgtaaatatcagtttttgttttggtttgttttttcgaggcagggtttctctgtatagccctggctgtcctggaactcacactgtagaccaggctggcctcgaactcaaaaatccgcttgcctctgcctcccgagtgctgggataaaaggcgcaccaccactgcccggcataagtagcagttttaattactccttaaatgcttctttattttcaataattgtgcagtacacgagggagtgagaaatgggttcggtgctgcagaggaactctggggcaggtatggagacctgaactcttgacagggggaacagatgcctcaagcagtggcgtgaggctctgcttgctgctctgcaggtgttttctattgcggtgttcggaccgattgtcaacgagggctacgtgaactccgacagtggtccagaactgcgctgcgtcttcaacggaaatgcgggagcctgccgcttcggcgtcgtactaggtctaggggccttcatcgcctgcgtggccttcttgctgctggacgtgcgctttcagcagatcagcagcgtccgagaccgtcgccgggctgtgctactggacctgggcttctcaggtgggcggggccagcggtgaaaggcggggcctaatgtagggtgcggtgctggtgcgggacctgatgcctgagcgaggggggcgtgggtgccctcagaggagagacagagcctgattcttttgggaaaaccgtaggatctttgtggggacgtgactgggcccctaaggaacaggactaggcgcggggactgccgcagagcttcttccggggctgtggcaggctgagaaatactcctcagggttttgagccgggaggacccaaccacaagagggtgtgatctagagatacagggtggaagtcttgagttgaaagcaccgagtcaaaagaaaaaaaaaaaaaaagccgggcgtggtggtgcacgcctttaatcccagcacttgggcggcagaggcaggtggatttctgagttcgaggccagcttggtctacaaagtgagttccaggacagccaaggctacagagagaaaccctgtctcaaaaaaacaaaaaaaaaaaaaagaaaagaaagaaagaaagaaagaaagaaagaaagaaagaaagaaagaaagaaagaaagaaagaaagaaagaaagaaagaaagaaagaaagaaagaaagaaagaaagaaagaaagaaagaaagaaagaaagaaagagaaagcacagaatctctttagggcacgccaggactagagaaagaggtggctgatgggcggagtaaggctgggcttcgaagggggcggggctttggaggtcatgtgaatggcaatttgctggtgaacctaagggacattgcactttctctgaatgcgcgaggtgggtcctgttggcggagcactatctgatcatcagctcccacctgcaggggtctggtccttcctgtggttcgtaggcttctgtttcctcaccaatcagtggcaacgaacaacgccagggccaggcaccgcgcaggctggagatgcggcgcgtgctgccatcgctttcagcttcttctccatcctcagctgggtgagtgagggacccgtgggtggcagggttgggggtgtctccaggttttccccagctgaccctgattccctcacccaggtggcgctcaccgtgaaggccctgcagcggttccgcctaggcaccgatatgtctctctttgccacagatcagctaggtactggggctgcccaggcctacccaggctacccagtgggcagtggtgtggagggtacagaaacataccagagcccgcccttcactgagaccctggacaccagctccaaagggtaccaggtgcctgcctattagtaggtgttaggctccggccagggctggaaggccacctcaccaccgtaggcctcaaggcctcctgagaccttttctgggtcctaccgtgccaaggacagagaaggtggccactatgggcctctgggaccaaggggagtagcctccctggtgcctggactgtccccacaaagttcccccctagccagagatctcagtcttaaggccctgagaagaaaactacttcttggagtggactggcctgggaggcattccttttatgggccagccaaccagggactcacctgtccactctaggtcagaagtccagctgcctcccagctcagggtgcaagggctctggggaccagggagagggagactaattgtacccttaggtggggattatggcccttgttcccagtcccatgtccctggctccacatggtggtccaccctgtccagaggctccctcccctcctcttacaggatccctagagcagctcagccctgtcggtgtcgtgattgtgattaagtcttcaggtctcatctcatagtgcccccataagccgcccctctctgtgtggctcctgcccaggtgtttctggccaggaaggtatagacaactatgggcctgggcacccgcccttcagtaagaccttccctagggaacagacagctgggtacctgggtgccaggcaagacaagactcttacaaggagaggactaaaggtctcatctgcccagggtgtcactggagagtcgttcctgtaaatggcttaaccttggcttcctgaaccagtccctccctagtctatcttctatccccttttaaggcccagaccaggtgtgggtccttgtgcatagcagactactcatacattgcttgaagctggcttttcacatcaagtcaaaaccaaactcgttgccatgtctcattcttccagacaaaatgcctccctgtggagttgcagttgaatgacaaccctgtacgttgtagcatagaccaatcatgtggatatttaagtgaacatgtttacaatttttgtatatatggatctattccttttcctctgaaagaacaatccatgattgtgtattttcagtgtcccctgttacaactgcaacttctttacaataaagactgtacgtgcattgaccatcagtaaggtcccctggacttattctttgtccctttccctcctgtgttgggg

SEQ ID NO: 2 - protein, 229 aa, Mus musculus
MEGASFGAGRAGAAFDPVSFARRPQTLLRVVSWVFSIAVFGPIVNEGYVNSDSGPELRCVFNGNAGACRFGVVLGLGAFIACVAFLLLDVRFQQISSVRDRRRAVLLDLGFSGVWSFLWFVGFCFLTNQWQRTTPGPGTAQAGDAARAAIAFSFFSILSWVALTVKALQRFRLGTDMSLFATDQLGTGAAQAYPGYPVGSGVEGTETYQSPPFTETLDTSSKGYQVPAY

SEQ ID NO: 3 - DNA, 4620 bp, Homo sapiens
  source 1..4620: mol_type = genomic DNA
cctacccctttactccctcacccaagtgcccttcccagaggagcggactcctcctgtctgtcctcccggctctagcaaagtctgcgcccagcacccgagccccaccctgcccccggggacctggctggtgggttcctgaggatggtctccatctcgggaccggggcaggcaggtgagggtgggggatgggaggtgggcgcggcggagggagaggagggacccggccccgcgcgcatggacccagtggggggcgcgggcgcggccccgccccgtcccgcgcgtccccgccgcggccggcgcgcgctcccgggaggcggcagcggctgcagcgttggtagcatcagcatcagcatcagcggcagcggcagcggcctcgggcggggccggccggacggacaggcggacagaaggcgccaggggcgcgcgtcccgcccgggccggccatggagggcgcctccttcggcgcgggccgcgcaggggccgccctggaccccgtgagctttgcgcggcggccccagaccctgctccgggtcgcgtcctgggtgagtggtccctgcccgggcccccgctcccgcccctgcctcgcgaccttcaggcccctaccagccccctgccccctaccccctgccccctgcttctcgccccccgacctcactcactctcatcctcgccggcccctcccccgccggcctcaggttggggtgacgtcaccgggcagggcgcgcccacctgcgggcggaggaggggccggcggcgccggagagggaccttgagaggtcaccgccggtcgcctctacccctacctcctccccgggtctaatttcagtccctttccgcagcccttactccgtttttcctgttctcgtgacctggaagcagggacggggtggggacggaattctccgaggggcaggagggggctacgggaaccgagaagcgcctccccttcccccgcacacacaccctcgggtctccttggcagggagcctgtcccctggcccccagttccagctgtgagttgagggaggagaggctctgggctgggagggcttcctggcggcggtgtggaaggcaggtttgggagcagcctagcccactggggcgtccctgggagggccctgctgctccttccctccggcaggggaggtggcagttgggtgccgagctctgggttttgtccaggtggcaacctctgggccagccgcacctcggcgcctgtcttggaggagggcggtgcccacggtggggcaggggctttggcctcccctgcggagtggctctgaccagaccgggaggcaggacgctgcgttttggtccgagcgcacgtcccgacttgtggcccactcttggggacaagtgcatgtcccggcttcccccttggctccactctcggagctggagcgggaaaggagcgaagggatgaggttgaggctggaggtcgtttcttggaaacacagggctgccccgtgcagcgctggttaaaatgactgcggtccccctcatgcctgtctcccggaactggtgggcaggaggcattgaggtttgacggaacctcagaggtcaacggtgtcatcttttcagcccaaacacttacaggtgatattaataatcagtcacgtgggggctgtcatccctcgggtacccaccacgtgcaggaagctgggtggacatgtctgtcccagcactgcatgagctgtgcccgtcaccctatttgcatgctagaaaacaggccagacagttcccaaccgcgcaggaagcaacagctccgctgcctccataccctccctcccgccccgctctgcctgctgcactctcacctccccttcgccgttccggctccagcctgggaatcgcgggcccaggtgaaggctcctgttcccacactcttgagtgggctctgaggggactccacgggcccacgcggtgcagagtacctggcttgaatcaaccccggcttttgtcagccatgtgatcccggacaagtcacttcacctgttggggtcccaatgtcccccgcatttataaagagaataagaacaatggcgatcccacggggactttctgaggatttggtgaggggacgcatgtaaagtggctgtttaacacaatgtctgggcatagtagatgctcactaaacggcccgtgttgtcaataattactaaatacgcgagggttcgggaaagaaagaggtgacaccgccccccacccagatacgggcctgggaacgcagggacaggcccaggggcgtgggcgctcgaggcgggctcgcagaggtcgggtcgccgcagggccctgagcgccgcgccgcacgcaggtgttctccatcgccgtcttcgggcccatcgtcaacgagggctacgtgaacaccgacagcggccccgagctgcgctgcgtgttcaacgggaacgcgggcgcctgccgcttcggcgtcgcgctgggcctcggagccttcctcgcctgcgccgccttcctgctgctcgatgtgcgcttccagcaaatcagcagcgtccgcgaccgccggcgcgcggtgttgctggacctgggcttctcaggtgggcggggccggggcggtgagcgcggagagccttccgggtgggcggggagggggcggggcctgggcggggaacaccgctggagtttccagctgggcgtggccgtgacgaggggcggggactgaggcagggagtgtcaatgggcctcccgggtgggcggggagggggcggagcctggacggggagcgccgcgggactttctaggtaggcggggcccgggtctgggcggagcctgggcgcggaacgggtctggcgctcccgggtgggcggggtcagcgcaggagagggaggcgggacctcgcgccacgcggcgagcccaggcgaggcgccccaagcctcgggcccaccgacctttcctcctccgggcgaggccgccgtgggccaccgcgtggagcgtcgccctgacgcgccgcactgttcgcaggactctggtccttcctgtggttcgtgggcttctgcttcctcaccaatcagtggcagcgcacggcgccagggccggccacgacgcaggcgggggacgcggcgcgggccgccatcgccttcagcttcttctccatcctcagctgggtgagtgcggggcccgggagggcggggcgaaggggcgggcgctcggctgatcccggctgaccccgctgaccccgccccgcgcaggtggcgctcaccgtgaaggccctgcagcggttccgcctgggcaccgacatgtcactcttcgccaccgaacagctgagcaccggggcgagccaggcctaccccggctatccggtgggcagcggcgtggagggcaccgagacctaccagagcccgcccttcaccgagaccctggacaccagccccaaagggtaccaggtgcccgcctactagcggctggcaggcacagaccagggctccaaggccaccccaccaacgcaggccccagggtctccgggacctcccttgggtccttccagctcagtgccgcggacagagtaggtggccgctttgcgccatccggggccaagagggggtggacccgcgtgtctgggctgcccctgccaagttcccccagtccctcagcacctggccccaggactgaggtcctgagaaggggatagcactgcccaggacgtgtgtccctagcctggaatggactggcctggggaaggctttcccctcttgggccacacctgctcactctggggttgggggtccagctgccctctacgatcaggtgcaggggctgcccaggacaaagcgggggcaggggaaagacaccaccctcgccccaagactggggatcctggccactgttcccatcccatgtccctgtgggtagtgactgtctcgtttctgtcatggtggtgcgtcccgtccggagccactctccactttctctcacaggctgctagaacagcccagccctgtcagtgttgtgatcatggtccagtcttcgggtttcacctcctagtactccacaagctgctcctctctctgtggccccggcccctgcccaggtgtgggtggttctggccaggaaggcacaaggtagctgtgggccaagacaccagccctgtcctagcccttcagtaagaccttgccaggagaggagaaggatgcctgggtgccaggcaagacaagcccctcagcaggagagaggcccagaggctccagctggccaccgtgccccacaagatggcccctgtgtggttccctttaccttggcttcctggcccagtccctgcctctccacctgcaccctgcttcctggcccagtcccaggttggagtccctctgcatagctgactactcatgcattgctcaaagctggcttttcacattaagtcaacaccaaacgtggttgccacatttcatcagacagacacctccctctggagatgcagttgagtgacaaccttgttacattgtagcctagaccaattctgtgtggatatttaagtgaacatgtttacaatttttgtatatatcactctctccctctcctgaaagaccagagattgtgtattttcagtgtcccatgttccgactgcaccttctttacaataaagactgtaactgagctgactgtgacctg

SEQ ID NO: 4 - protein, 229 aa, Homo sapiens
MEGASFGAGRAGAALDPVSFARRPQTLLRVASWVFSIAVFGPIVNEGYVNTDSGPELRCVFNGNAGACRFGVALGLGAFLACAAFLLLDVRFQQISSVRDRRRAVLLDLGFSGLWSFLWFVGFCFLTNQWQRTAPGPATTQAGDAARAAIAFSFFSILSWVALTVKALQRFRLGTDMSLFATEQLSTGASQAYPGYPVGSGVEGTETYQSPPFTETLDTSPKGYQVPAY

SEQ ID NO: 5 - DNA, 17 bp, synthetic construct
  source 1..17: mol_type = other DNA
gcactttgccttctgtc

SEQ ID NO: 6 - DNA, 16 bp, synthetic construct
  source 1..16: mol_type = other DNA
cgcaaagctcacggga

SEQ ID NO: 7 - DNA, 17 bp, synthetic construct
  source 1..17: mol_type = other DNA
gccgcgcaaagctcacg

SEQ ID NO: 8 - DNA, 16 bp, synthetic construct
  source 1..16: mol_type = other DNA
caactaaactccttga

SEQ ID NO: 9 - DNA, 16 bp, synthetic construct
  source 1..16: mol_type = other DNA
atcattccacaattct

SEQ ID NO: 10 - DNA, 16 bp, synthetic construct
  source 1..16: mol_type = other DNA
gcaaaccacagtcatt

SEQ ID NO: 11 - DNA, 16 bp, synthetic construct
  source 1..16: mol_type = other DNA
gattcccgtgagtagt

SEQ ID NO: 12 - DNA, 16 bp, synthetic construct
  source 1..16: mol_type = other DNA
aacaattgatcgggag

SEQ ID NO: 13 - DNA, 17 bp, synthetic construct
  source 1..17: mol_type = other DNA
tcggtgaagggcgggct

SEQ ID NO: 14 - DNA, 17 bp, synthetic construct
  source 1..17: mol_type = other DNA
cgtagccctcgttgaca

SEQ ID NO: 15 - DNA, 20 bp, synthetic construct
  source 1..20: mol_type = other DNA
ttcacgtagccctcgttgac

SEQ ID NO: 16 - DNA, 20 bp, synthetic construct
  source 1..20: mol_type = other DNA
gttcacgtagccctcgttga

SEQ ID NO: 17 - DNA, 20 bp, synthetic construct
  source 1..20: mol_type = other DNA
gttcacgtagccctcgttga

SEQ ID NO: 18 - DNA, 17 bp, synthetic construct
  source 1..17: mol_type = other DNA
tcacgtagccctcgttg

SEQ ID NO: 19 - DNA, 20 bp, synthetic construct
  source 1..20: mol_type = other DNA
gtgttcacgtagccctcgtt

SEQ ID NO: 20 - DNA, 20 bp, synthetic construct
  source 1..20: mol_type = other DNA
acgaaccacaggaaggacca

SEQ ID NO: 21 - DNA, 16 bp, synthetic construct
  source 1..16: mol_type = other DNA
cgttgccactgattgg

SEQ ID NO: 22 - DNA, 20 bp, synthetic construct
  source 1..20: mol_type = other DNA
agctgaggatggagaagaag

SEQ ID NO: 23 - DNA, 17 bp, synthetic construct
  source 1..17: mol_type = other DNA
cagctgaggatggagaa

SEQ ID NO: 24 - DNA, 17 bp, synthetic construct
  source 1..17: mol_type = other DNA
ggccttcacggtgagcg

SEQ ID NO: 25 - DNA, 17 bp, synthetic construct
  source 1..17: mol_type = other DNA
tctgtggcaaagagaga

SEQ ID NO: 26 - DNA, 17 bp, synthetic construct
  source 1..17: mol_type = other DNA
ggcgggctctggtaggt

SEQ ID NO: 27 - DNA, 17 bp, synthetic construct
  source 1..17: mol_type = other DNA
aagggcgggctctggta

SEQ ID NO: 28 - DNA, 20 bp, synthetic construct
  source 1..20: mol_type = other DNA
gtgaagggcgggctctggta

SEQ ID NO: 29 - DNA, 17 bp, synthetic construct
  source 1..17: mol_type = other DNA
gtgaagggcgggctctg

SEQ ID NO: 30 - DNA, 17 bp, synthetic construct
  source 1..17: mol_type = other DNA
tcggtgaagggcgggct

SEQ ID NO: 31 - DNA, 17 bp, synthetic construct
  source 1..17: mol_type = other DNA
agactagggagggactg

SEQ ID NO: 32 - DNA, 17 bp, synthetic construct
  source 1..17: mol_type = other DNA
ctagggagggactggtt

SEQ ID NO: 33 - DNA, 17 bp, synthetic construct
  source 1..17: mol_type = other DNA
agatagactagggaggg

SEQ ID NO: 34 - DNA, 17 bp, synthetic construct
  source 1..17: mol_type = other DNA
ggatagaagatagacta

SEQ ID NO: 35 - DNA, 17 bp, synthetic construct
  source 1..17: mol_type = other DNA
agacatggcaacgagtt

SEQ ID NO: 36 - DNA, 17 bp, synthetic construct
  source 1..17: mol_type = other DNA
gtctggaagaatgagac

SEQ ID NO: 37 - DNA, 17 bp, synthetic construct
  source 1..17: mol_type = other DNA
taaagaagttgcagttg

SEQ ID NO: 38 - DNA, 16 bp, Mus musculus
  source 1..16: mol_type = genomic DNA
tcccgtgagctttgcg

SEQ ID NO: 39 - DNA, 17 bp, Mus musculus
  source 1..17: mol_type = genomic DNA
cgtgagctttgcgcggc

SEQ ID NO: 40 - DNA, 16 bp, Mus musculus
  source 1..16: mol_type = genomic DNA
agaattgtggaatgat

SEQ ID NO: 41 - DNA, 16 bp, Mus musculus
  source 1..16: mol_type = genomic DNA
aatgactgtggtttgc

SEQ ID NO: 42 - DNA, 16 bp, Mus musculus
  source 1..16: mol_type = genomic DNA
actactcacgggaatc

SEQ ID NO: 43 - DNA, 17 bp, Mus musculus
  source 1..17: mol_type = genomic DNA
tgtcaacgagggctacg

SEQ ID NO: 44 - DNA, 20 bp, Mus musculus
  source 1..20: mol_type = genomic DNA
tcaacgagggctacgtgaac

SEQ ID NO: 45 - DNA, 17 bp, Mus musculus
  source 1..17: mol_type = genomic DNA
caacgagggctacgtga

SEQ ID NO: 46 - DNA, 16 bp, Mus musculus
  source 1..16: mol_type = genomic DNA
ccaatcagtggcaacg

SEQ ID NO: 47 - DNA, 17 bp, Mus musculus
  source 1..17: mol_type = genomic DNA
ttctccatcctcagctg

SEQ ID NO: 48 - DNA, 17 bp, Mus musculus
  source 1..17: mol_type = genomic DNA
cgctcaccgtgaaggcc

SEQ ID NO: 49 - DNA, 17 bp, Mus musculus
  source 1..17: mol_type = genomic DNA
tctctctttgccacaga

SEQ ID NO: 50 - DNA, 17 bp, Mus musculus
  source 1..17: mol_type = genomic DNA
tagtctatcttctatcc

SEQ ID NO: 51 - DNA, 17 bp, Mus musculus
  source 1..17: mol_type = genomic DNA
gtctcattcttccagac

SEQ ID NO: 52 - DNA, 20 bp, Mus musculus
  source 1..20: mol_type = genomic DNA
tcccgtgagctttgcgcggc

SEQ ID NO: 53 - DNA, 19 bp, Mus musculus
  source 1..19: mol_type = genomic DNA
cccgtgagctttgcgcggc

SEQ ID NO: 54 - DNA, 22 bp, Mus musculus
  source 1..22: mol_type = genomic DNA
tgtcaacgagggctacgtgaac

SEQ ID NO: 55 - DNA, 16 bp, synthetic construct
  source 1..16: mol_type = other DNA
gcgcaaagctcacggg

SEQ ID NO: 56 - DNA, 16 bp, synthetic construct
  source 1..16: mol_type = other DNA
cgcgcaaagctcacgg

SEQ ID NO: 57 - DNA, 16 bp, synthetic construct
  source 1..16: mol_type = other DNA
ccgcgcaaagctcacg

SEQ ID NO: 58 - DNA, 16 bp, synthetic construct
  source 1..16: mol_type = other DNA
gccgcgcaaagctcac

SEQ ID NO: 59 - DNA, 16 bp, synthetic construct
  source 1..16: mol_type = other DNA
cgccgcgcaaagctca

SEQ ID NO: 60 - DNA, 16 bp, synthetic construct
  source 1..16: mol_type = other DNA
acgtagccctcgttga

SEQ ID NO: 61 - DNA, 16 bp, synthetic construct
  source 1..16: mol_type = other DNA
gttcacgtagccctcg

SEQ ID NO: 62 - DNA, 16 bp, synthetic construct
  source 1..16: mol_type = other DNA
tgagcgccacctgcgc

SEQ ID NO: 63 - DNA, 16 bp, synthetic construct
  source 1..16: mol_type = other DNA
acggtgagcgccacct

SEQ ID NO: 64 - DNA, 16 bp, synthetic construct
  source 1..16: mol_type = other DNA
tcacggtgagcgccac

SEQ ID NO: 65 - DNA, 16 bp, synthetic construct
  source 1..16: mol_type = other DNA
ttcacggtgagcgcca

SEQ ID NO: 66 - DNA, 16 bp, synthetic construct
  source 1..16: mol_type = other DNA
cttcacggtgagcgcc

SEQ ID NO: 67 - DNA, 16 bp, synthetic construct
  source 1..16: mol_type = other DNA
gcggaaccgctgcagg

SEQ ID NO: 68 - DNA, 16 bp, synthetic construct
  source 1..16: mol_type = other DNA
cccaggcggaaccgct

SEQ ID NO: 69 - DNA, 42 bp, Homo sapiens
  source 1..42: mol_type = genomic DNA
ccgcgcaggggccgccctggaccccgtgagctttgcgcggcg

SEQ ID NO: 70 - DNA, 32 bp, Homo sapiens
  source 1..32: mol_type = genomic DNA
gccgccctggaccccgtgagctttgcgcggcg

SEQ ID NO: 71 - DNA, 27 bp, Homo sapiens
  source 1..27: mol_type = genomic DNA
cctggaccccgtgagctttgcgcggcg

SEQ ID NO: 72 - DNA, 22 bp, Homo sapiens
  source 1..22: mol_type = genomic DNA
accccgtgagctttgcgcggcg

SEQ ID NO: 73 - DNA, 20 bp, Homo sapiens
  source 1..20: mol_type = genomic DNA
cccgtgagctttgcgcggcg

SEQ ID NO: 74 - DNA, 25 bp, Homo sapiens
  source 1..25: mol_type = genomic DNA
cgtcaacgagggctacgtgaacacc

SEQ ID NO: 75 - DNA, 21 bp, Homo sapiens
  source 1..21: mol_type = genomic DNA
tcaacgagggctacgtgaaca

SEQ ID NO: 76 - DNA, 67 bp, Homo sapiens
  source 1..67: mol_type = genomic DNA
tcccggctgaccccgctgaccccgccccgcgcaggtggcgctcaccgtgaaggccctgcagcggttc

SEQ ID NO: 77 - DNA, 57 bp, Homo sapiens
  source 1..57: mol_type = genomic DNA
ccccgctgaccccgccccgcgcaggtggcgctcaccgtgaaggccctgcagcggttc

SEQ ID NO: 78 - DNA, 52 bp, Homo sapiens
  source 1..52: mol_type = genomic DNA
ctgaccccgccccgcgcaggtggcgctcaccgtgaaggccctgcagcggttc

SEQ ID NO: 79 - DNA, 49 bp, Homo sapiens
  source 1..49: mol_type = genomic DNA
accccgccccgcgcaggtggcgctcaccgtgaaggccctgcagcggttc

SEQ ID NO: 80 - protein, 11 aa, synthetic construct
YGRKKRRQRRR

SEQ ID NO: 81 - protein, 18 aa, synthetic construct
KLALKLALKALKAALKLA

SEQ ID NO: 82 - protein, 16 aa, synthetic construct
RQIKIWFQNRRMKWKK

SEQ ID NO: 83 - DNA, 16 bp, synthetic construct
  source 1..16: mol_type = other DNA
ccttctgtccgcctgt

SEQ ID NO: 84 - DNA, 16 bp, synthetic construct
  source 1..16: mol_type = other DNA
cgccttctgtccgcct

SEQ ID NO: 85 - DNA, 16 bp, synthetic construct
  source 1..16: mol_type = other DNA
cgaaggaggcgccctc

SEQ ID NO: 86 - DNA, 16 bp, synthetic construct
  source 1..16: mol_type = other DNA
ccgaaggaggcgccct

SEQ ID NO: 87 - DNA, 16 bp, synthetic construct
  source 1..16: mol_type = other DNA
ccgccgcgcaaagctc

SEQ ID NO: 88 - DNA, 16 bp, synthetic construct
  source 1..16: mol_type = other DNA
gccgccgcgcaaagct

SEQ ID NO: 89 - DNA, 16 bp, synthetic construct
  source 1..16: mol_type = other DNA
tagccctcgttgacga

SEQ ID NO: 90 - DNA, 16 bp, synthetic construct
  source 1..16: mol_type = other DNA
ggtgttcacgtagccc

SEQ ID NO: 91 - DNA, 16 bp, synthetic construct
  source 1..16: mol_type = other DNA
cggtgttcacgtagcc

SEQ ID NO: 92 - DNA, 16 bp, synthetic construct
  source 1..16: mol_type = other DNA
ggatcagccgagcgcc

SEQ ID NO: 93 - DNA, 16 bp, synthetic construct
  source 1..16: mol_type = other DNA
ccgggatcagccgagc

SEQ ID NO: 94 - DNA, 16 bp, synthetic construct
  source 1..16: mol_type = other DNA
agccgggatcagccga

SEQ ID NO: 95 - DNA, 16 bp, synthetic construct
  source 1..16: mol_type = other DNA
tgcccaggcggaaccg

SEQ ID NO: 96 - DNA, 16 bp, synthetic construct
  source 1..16: mol_type = other DNA
acaggcggacagaagg

SEQ ID NO: 97 - DNA, 16 bp, synthetic construct
  source 1..16: mol_type = other DNA
aggcggacagaaggcg

SEQ ID NO: 98 - DNA, 16 bp, synthetic construct
  source 1..16: mol_type = other DNA
gagggcgcctccttcg

SEQ ID NO: 99 - DNA, 16 bp, synthetic construct
  source 1..16: mol_type = other DNA
agggcgcctccttcgg

SEQ ID NO: 100 - DNA, 16 bp, synthetic construct
  source 1..16: mol_type = other DNA
cccgtgagctttgcgc

SEQ ID NO: 101 - DNA, 16 bp, synthetic construct
  source 1..16: mol_type = other DNA
ccgtgagctttgcgcg

SEQ ID NO: 102 - DNA, 16 bp, synthetic construct
  source 1..16: mol_type = other DNA
cgtgagctttgcgcgg

SEQ ID NO: 103 - DNA, 16 bp, synthetic construct
  source 1..16: mol_type = other DNA
gtgagctttgcgcggc

SEQ ID NO: 104 - DNA, 16 bp, synthetic construct
  source 1..16: mol_type = other DNA
tgagctttgcgcggcg

SEQ ID NO: 105 - DNA, 16 bp, synthetic construct
  source 1..16: mol_type = other DNA
gagctttgcgcggcgg

SEQ ID NO: 106 - DNA, 16 bp, synthetic construct
  source 1..16: mol_type = other DNA
agctttgcgcggcggc

SEQ ID NO: 107 - DNA, 16 bp, synthetic construct
  source 1..16: mol_type = other DNA
tcgtcaacgagggcta

SEQ ID NO: 108 - DNA, 16 bp, synthetic construct
  source 1..16: mol_type = other DNA
tcaacgagggctacgt

SEQ ID NO: 109 - DNA, 16 bp, synthetic construct
  source 1..16: mol_type = other DNA
cgagggctacgtgaac

SEQ ID NO: 110 - DNA, 16 bp, synthetic construct
  source 1..16: mol_type = other DNA
gggctacgtgaacacc

SEQ ID NO: 111 - DNA, 16 bp, synthetic construct
  source 1..16: mol_type = other DNA
ggctacgtgaacaccg

SEQ ID NO: 112 - DNA, 16 bp, synthetic construct
  source 1..16: mol_type = other DNA
ggcgctcggctgatcc

SEQ ID NO: 113 - DNA, 16 bp, synthetic construct
  source 1..16: mol_type = other DNA
gctcggctgatcccgg

SEQ ID NO: 114 - DNA, 16 bp, synthetic construct
  source 1..16: mol_type = other DNA
tcggctgatcccggct

SEQ ID NO: 115 - DNA, 16 bp, synthetic construct
  source 1..16: mol_type = other DNA
gcgcaggtggcgctca

SEQ ID NO: 116 - DNA, 16 bp, synthetic construct
  source 1..16: mol_type = other DNA
aggtggcgctcaccgt

SEQ ID NO: 117 - DNA, 16 bp, synthetic construct
  source 1..16: mol_type = other DNA
gtggcgctcaccgtga

SEQ ID NO: 118 - DNA, 16 bp, synthetic construct
  source 1..16: mol_type = other DNA
tggcgctcaccgtgaa

SEQ ID NO: 119 - DNA, 16 bp, synthetic construct
  source 1..16: mol_type = other DNA
ggcgctcaccgtgaag

SEQ ID NO: 120 - DNA, 16 bp, synthetic construct
  source 1..16: mol_type = other DNA
cctgcagcggttccgc

SEQ ID NO: 121 - DNA, 16 bp, synthetic construct
  source 1..16: mol_type = other DNA
agcggttccgcctggg

SEQ ID NO: 122 - DNA, 16 bp, synthetic construct
  source 1..16: mol_type = other DNA
cggttccgcctgggca

SEQ ID NO: 123 - protein, 4 aa, synthetic construct
AAAA

(19) United States
(12) Patent Application Publication
(10) Pub. No.: US 2025/0011793 A1
(43) Pub. Date: Jan. 9, 2025

(54) Oligonucleotides for Modulating Synaptogyrin-3 Expression

(71) Applicants: VIB VZW, Gent (BE); Katholieke Universiteit Leuven K.u.Leuven R&D, Leuven (BE); Jay Therapeutics, Gent (BE)

(72) Inventors: Patrik Verstreken, Blanden (BE); Ana Rita Santos, Herent (BE)

(21) Appl. No.: 18/684,059

(22) PCT Filed: Aug. 16, 2022
(86) PCT No.: PCT/EP2022/072878

§ 371 Date: Feb. 15, 2024

(30) Foreign Application Priority Data
Aug. 16, 2021 (EP) 21191496.5

(51) Int. Cl. C12N 15/113

(52) U.S. Cl. CPC C12N 15/1138; C12N 2310/315; C12N 2310/32; C12N 2310/341

(57) ABSTRACT

The invention relates to identification of regions within the synaptogyrin-3 RNA sequence that are targetable by oligonucleotide inhibitors. In particular, these synaptogyrin-3 inhibitors are provided for use as a medicament in general, and for treating or inhibiting progression of tauopathies or symptoms of tauopathies.

### CROSS REFERENCE TO RELATED APPLICATIONS

This application is a national phase entry under 35 U.S.C. § 371 of International Patent Application PCT/EP2022/072878, filed Aug. 16, 2022, designating the United States of America and published in English as International Patent Publication WO 2023/021046 on Feb. 23, 2023, which claims the benefit under Article 8 of the Patent Cooperation Treaty to European Patent Application Serial No. 21191496.5, filed Aug. 16, 2021, the entireties of which are hereby incorporated by reference.

### INCORPORATION BY REFERENCE

The ST.26 XML Sequence listing named “10343USSequenceListingST26”, created on Jan. 22, 2024, and having a size of 122,880 bytes, is hereby incorporated herein by this reference in its entirety.

### FIELD OF THE INVENTION

The invention relates to identification of regions within the synaptogyrin-3 RNA sequence that are targetable by oligonucleotide inhibitors. In particular, these synaptogyrin-3 inhibitors are provided for use as a medicament in general, and for treating or inhibiting progression of tauopathies or symptoms of tauopathies in particular.

### BACKGROUND TO THE INVENTION

Tau pathology is associated with more than twenty neurodegenerative diseases, including Alzheimer's disease (Wang & Mandelkow 2016 Nat Rev Neurosci 17:5-21). Hyperphosphorylation or mutation of the microtubule-associated protein Tau is common to all of these diseases, collectively termed Tauopathies, and filamentous inclusions of hyperphosphorylated Tau are hallmark pathologies of Alzheimer's disease and other Tauopathies (Ballatore et al 2007 Nature Reviews Neuroscience 8:663-672). Tau pathology is not merely a byproduct of other pathological pathways, but is a key mediator of neurotoxicity itself (Roberson et al 2007 Science 316:750-754; Hutton et al 1998 Nature 393:702-705; Caffrey & Wade-Martins 2007 Neurobiol Dis 27:1-10; Le Guennec et al 2016 Molecular Psychiatry 1-7). Under physiological conditions, Tau is expressed in neurons and is bound to axonal microtubules. However, under pathological conditions, mutations in Tau (in FTDP-17) or abnormal phosphorylation of Tau (including sporadic Alzheimer's disease) decrease its microtubule binding affinity (Hong et al 1998 Science 282:1914-1917; Wang & Mandelkow 2016 Nat Rev Neurosci 17:5-21), leading to its dissociation from axonal microtubules and subsequent mislocalization to synapses (Spires-Jones & Hyman 2014 Neuron 82:756-771; Tai et al 2012 Am J Pathol 181:1426-1435; Tai et al 2014 Acta Neuropathol Commun 2:146). This mislocalisation of soluble Tau plays a key role in perturbing synaptic function in early disease stages, which may contribute to subsequent synapse loss and neurodegeneration.

In addition to the reported post-synaptic localization of pathological Tau (Hoover et al 2010 Neuron 68:1067-1081; Ittner et al 2010 Cell 142:387-397; Zhao et al 2016 Nat Med 22:1268-1276), it was previously shown that hyperphosphorylated Tau species accumulate on presynaptic vesicles isolated from Alzheimer's disease patient brain. This suggests that the presynaptic pathway also contributes to synaptic dysfunction in human neurodegenerative diseases associated with Tau. Using unbiased proteomic and genetic approaches, it was found that the transmembrane synaptic vesicle protein Synaptogyrin-3 mediates the association of Tau with synaptic vesicles in vitro and in vivo (WO2019/016123). Reduction of Drosophila Synaptogyrin or murine Synaptogyrin-3 levels in neurons from fly and mouse models of tauopathy reduced the association of Tau with synaptic vesicles, and subsequently rescued Tau-induced defects in vesicle mobility and neurotransmitter release. These findings identify Synaptogyrin-3 as a novel Tau interactor that mediates synaptic dysfunction associated with Tau (WO2019/016123). It would thus be advantageous to develop specific Synaptogytin-3 inhibitors for use in treating various Tauopathies including Alzheimer's disease, conditions for which there continues to be a high unmet need for therapies. Impacting Tau-mediated pathways at the presynapse is highly relevant given that neurodegeneration is thought to begin with loss of presynaptic terminals and proceed retrograde in a dyeing-back process (Yoshiyama et al 2007 Neuron 53:337-351).

### SUMMARY OF THE INVENTION

In one aspect, the invention relates to oligonucleotides that specifically bind to Synaptogyrin-3 transcript and reduce the expression of Synaptogyrin-3 through antisense or RNAi technology.

More particularly, an oligonucleotide of 10 to 50 nucleotides in length is provided comprising a contiguous nucleotide sequence of at least 10 contiguous nucleotides in length which are at least 90% complementary to an equal length portion of nucleotides 234 to 253, 1144 to 1159, 1205 to 1220, 1319 to 1334, 2271 to 2287, 2295 to 2316, 3387 to 3402, 3470 to 3486, 3570 to 3586, 3617 to 3633, 4561 to 4577 or 4683 to 4699 of SEQ ID No. 1 or at least 90% complementary to an equal length portion of nucleotides 470 to 511, 2344 to 2368 or 3170 to 3236 of SEQ ID No. 3 In one embodiment, said contiguous nucleotide sequence is at least 12, 14 or 16 nucleotides in length.

In another embodiment, said contiguous nucleotide sequence is 100% complementary to a contiguous nucleotide sequence of equal length that is part of or comprised within the sequence set forth in any of SEQ ID No. 38-54, SEQ ID No. 69-79, SEQ ID No. 100-104, SEQ ID No. 108-109 or SEQ ID No. 115-121.

In another embodiment, said oligonucleotide comprises one or more internucleoside linkage and/or one or more 2′ sugar modified nucleosides. More particularly, the internucleoside linkage is a phosphorothioate internucleoside linkage and/or the 2′ sugar modified nucleoside is selected from the group consisting of 2′-O-methyl-, 2′-O-methoxyethyl-, 2′-O-alkyl-, 2′-alkoxy, 2′-amino-, 2′-fluoro- and LNA nucleosides. In another embodiment, said oligonucleotide is a single stranded antisense oligonucleotide, an siRNA, a shRNA, a CRISPR gRNA or forms the guide strand of an siRNA or shRNA complex.

In a particular embodiment, the oligonucleotide comprises a gapmer of formula 5′-F-G-F′-3′, where region F and F′ independently comprise between 1 and 8 nucleosides, of which 1 to 5 independently are 2′ sugar modified nucleosides and define the 5′ and 3′ end of the F and F′ region, and G is a region between 5 and 18 nucleosides for recruiting RNaseH. Even more particular, said oligonucleotide is provided wherein the internucleoside linkages between one or more nucleosides of region F and/or F′ and/or between F and G and/or between F′ and G are phosphorothioate internucleoside linkages.

In another aspect, a pharmaceutical composition comprising the oligonucleotide according to the invention is provided.

In yet another aspect, any oligonucleotide herein described or pharmaceutical composition comprising it is provided for use as a medicament. In particular, for use in (a method for) treating or inhibiting progression of a tauopathic disorder or for use in (a method for) treating or inhibiting a symptom of a tauopathic disorder. Regarding current application, the tauopathic disorder may be selected from the group consisting of Alzheimer's disease, progressive supranuclear palsy (PSP), progressive supranuclear palsy-parkinsonism (PSP-P), Richardson's syndrome, argyrophilic grain disease, corticobasal degeneration Pick's disease, frontotemporal dementia with parkinsonism associated with chromosome 17 (FTDP-17), post-encephalitic parkinsonism, Parkinson's disease complex of Guam, Guadeloupean parkinsonism, Huntington disease, Down's syndrome, dementia pugilistica, familial British dementia, familial Danish dementia, myotonic dystrophy, Hallevorden-Spatz disease, Niemann Pick type C, chronic traumatic encephalopathy, tangle-only dementia, white matter tauopathy with globular glial inclusions, subacute sclerosing panencephalitis, SLC9A6-related mental retardation, non-Guamanian motor neuron disease with neurofibrillary tangles, neurodegeneration with brain iron accumulation, Gerstmann-Straussler-Scheinker disease, frontotemporal lobar degeneration, diffuse neurofibrillary tangles with calcification, chronic traumatic encephalopathy, amyotrophic lateral sclerosis of Guam, amyotrophic lateral sclerosis and parkinsonism-dementia complex, prion protein cerebral amyloid angiopathy, and progressive subcortical gliosis.

In current application, the symptom of the tauopathic disorder may be selected from the group of mild cognitive impairment, dementia, cognitive decline, decline of motor function, oculomotor and bulbar dysfunction, synaptic dysfunction, neurotoxicity, neuronal degeneration, neuronal dysfunction, synapse loss, and amyloid deposition. The synaptic dysfunction may be further specified as pre-synaptic dysfunction.

### BRIEF DESCRIPTION OF THE FIGURES

FIG. 1 illustrates the strong reduction of SYNGR-3 transcript over time, after administration of VIB_017 and VIB_018. VIB_23 was used as a negative control. Primary neurons were incubated with 1.5 μM of ASOs for 24 h, 48 h, 72 h and 96 h. Neurons were lysed and the sample processed for qPCR analysis. A similar effect was observed in a different cellular background.

FIG. 2 shows that none of the tested treatments (VIB_17, VIB_18, VIB_23 and mock) had a negative effect in cell viability as measured by the levels of ATP (ATPlite). This effect was observed in different cellular background.

FIG. 3 shows a dose-response curve of VIB_017 and VIB_018 on the Syngr3 mRNA expression level in primary neurons. The non-targeting ASO was used as control and did not alter Syngr3 expression. A top concentration of 2 μM was used with 7 additional steps of a 3-fold dilution/step. Primary neurons were incubated at day 7 with the relevant ASOs for 96 h in a free-uptake mode. Previous experiments show that the expression of Syngr3 in cortical primary neurons is stable during the period of the treatment.

### DETAILED DESCRIPTION OF THE INVENTION

In one aspect, the current invention relates to oligonucleotides (“oligonucleotides of the present disclosure”) that specifically bind to Synaptogyrin-3 RNA and reduce the expression of Synaptogyrin-3, e.g. through antisense or RNAi technology. In some aspects, the oligonucleotide of the present disclosure is 10 to 50, 10 to 40, or of 10 to 30 nucleotides in length, and comprises a contiguous nucleotide sequence of at least 10 contiguous nucleotides in length which are at least 90% complementary to a region of mSynaptogyrin-3 selected from the group consisting of TCCCGTGAGCTTTGCG (SEQ ID No. 38), CGTGAGCTTTGCGCGGC (SEQ ID No. 39), AGAATTGTGGAATGAT (SEQ ID No. 40), AATGACTGTGGTTTGC (SEQ ID No. 41), ACTACTCACGGGAATC (SEQ ID No. 42), TGTCAACGAGGGCTACG (SEQ ID No. 43), TCAACGAGGGCTACGTGAAC (SEQ ID No. 44), CAACGAGGGCTACGTGA (SEQ ID No. 45), CCAATCAGTGGCAACG (SEQ ID No. 46), TTCTCCATCCTCAGCTG (SEQ ID No. 47), CGCTCACCGTGAAGGCC (SEQ ID No. 48), TCTCTCTTTGCCACAGA (SEQ ID No. 49), TAGTCTATCTTCTATCC (SEQ ID No. 50) and GTCTCATTCTTCCAGAC (SEQ ID No. 51). In some aspects, the oligonucleotide of the present disclosure is 10 to 50, 10 to 40, or 10 to 30 nucleotides in length, and comprises a contiguous nucleotide sequence of at least 10 contiguous nucleotides in length which are at least 90% complementary to region 234-253 of SEQ ID No. 1 (i.e. TCCCGTGAGCTTTGCGCGGC or SEQ ID No. 52), region 235-253 of SEQ ID No. 1 (i.e. CCCGTGAGCTTTGCGCGGC or SEQ ID No. 53) or to region 2295-2316 of SEQ ID No. 1 (i.e. TGTCAACGAGGGCTACGTGAAC or SEQ ID No. 54). In some aspects, the oligonucleotide of the present disclosure is 10 to 50, 10 to 40, or of 10 to 30 nucleotides in length, and comprises a contiguous nucleotide sequence of at least 10 contiguous nucleotides in length which are at least 90% complementary to a region of human Synaptogyrin-3 selected from the group consisting of

(SEQ ID No. 100)
CCCGTGAGCTTTGCGC,
(SEQ ID No. 101)
CCGTGAGCTTTGCGCG,
(SEQ ID No. 102)
CGTGAGCTTTGCGCGG,
(SEQ ID No. 103)
GTGAGCTTTGCGCGGC,
(SEQ ID No. 104)
TGAGCTTTGCGCGGCG,
(SEQ ID No. 108)
TCAACGAGGGCTACGT,
(SEQ ID No. 109)
CGAGGGCTACGTGAAC,
(SEQ ID No. 115)
GCGCAGGTGGCGCTCA,
(SEQ ID No. 116)
AGGTGGCGCTCACCGT,
(SEQ ID No. 117)
GTGGCGCTCACCGTGA,
(SEQ ID No. 118)
TGGCGCTCACCGTGAA,
(SEQ ID No. 119)
GGCGCTCACCGTGAAG,
(SEQ ID No. 120)
CCTGCAGCGGTTCCGC,
(SEQ ID NO. 121)
AGCGGTTCCGCCTGGG.

In some aspects, the oligonucleotide of the present disclosure is 10 to 50, 10 to 40, or 10 to 30 nucleotides in length, and comprises a contiguous nucleotide sequence of at least 10 contiguous nucleotides in length which are at least 90% complementary to region 470-511 of SEQ ID No.3 (i.e. CCGCGCAGGGGCCGCCCTGGACCCCGTGAGCTTTGCGCGGCG or SEQ ID No. 69), to region 480-511 of SEQ ID No. 3 (i.e. GCCGCCCTGGACCCCGTGAGCTTTGCGCGGCG or SEQ ID No. 70), to region 485-511 of SEQ ID No. 3 (i.e. CCTGGACCCCGTGAGCTTTGCGCGGCG or SEQ ID No. 71), to region 490-511 of SEQ ID No. 3 (i.e. ACCCCGTGAGCTTTGCGCGGCG or SEQ ID No. 72), to region 492-511 of SEQ ID No. 3 (i.e. CCCGTGAGCTTTGCGCGGCG or SEQ ID No. 73), to region 2344-2368 of SEQ ID No. 3 (i.e. CGTCAACGAGGGCTACGTGAACACC or SEQ ID No. 74), to region 2346-2366 of SEQ ID No. 3 (i.e. TCAACGAGGGCTACGTGAACA of SEQ ID No. 75), to region 3170-3236 of SEQ ID No. 3 (i.e. TCCCGGCTGACCCCGCTGACCCCGCCCCGCGCAGGTGGCGCTCACCGTGAAGGCCCTGCAGCGGTTC or SEQ ID No. 76), to region 3180-3236 of SEQ ID No. 3 (i.e. CCCCGCTGACCCCGCCCCGCGCAGGTGGCGCTCACCGTGAAGGCCCTGCAGCGGTTC or SEQ ID No. 77), to region 3185-3236 of SEQ ID No. 3 (i.e. CTGACCCCGCCCCGCGCAGGTGGCGCTCACCGTGAAGGCCCTGCAGCGGTTC or SEQ ID No. 78) or to region 3188-3236 of SEQ ID No. 3 (i.e. ACCCCGCCCCGCGCAGGTGGCGCTCACCGTGAAGGCCCTGCAGCGGTTC or SEQ ID No. 79).

In some aspects, the oligonucleotide of the present disclosure is complementary (full or partially complementary) to a target region of Synaptogyrin-3 selected from the group consisting of SEQ ID No. 38-54, SEQ ID No. 69-79, SEQ ID No. 100-104, SEQ ID No. 108-109 and SEQ ID No. 115-121.

In some aspects, the oligonucleotide of the present disclosure comprises or consists of 10, 11, 12, 13, 14, or 15 nucleotides, the oligonucleotide being a fragment or a part of the sequence set for in SEQ ID No. 7, 16-18, 24, 55-68. In some aspects, the oligonucleotide comprises or consists of 16, 17, 18, 19 or 20 nucleotides in length and comprises or consists of the sequence set for in SEQ ID No. 7, 16-18, 24, 55-68. In some aspects, the oligonucleotide of the present disclosure is 14 to 20 nucleotides in length, e.g., 14, 15, 16, 17, 18, 19, or 20 nucleotides in length. The present disclosure also provides methods of treatment comprising the administration of the oligonucleotides of the present disclosure, or a combination thereof, to a subject in need thereof. Also provides are pharmaceutical compositions, pharmaceutical formulations, and kits and articles of manufacture comprising the oligonucleotides of the present disclosure. Also provided are methods of manufacture of the oligonucleotides of the present disclosure.

### Definitions

In order that the present description can be more readily understood, certain terms are first defined. Additional definitions are set forth throughout the detailed description. The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

It is to be noted that the term “a” or “an” entity refers to one or more of that entity; for example, “a nucleotide sequence”, is understood to represent one or more nucleotide sequences. As such, the terms “a” (or “an”), “one or more” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

It is understood that wherever aspects are described herein with the language “comprising”, otherwise analogous aspects described in terms of “consisting of” and/or “consisting essentially of” are also provided. Where the term “comprising” is used in the present description and claims, it does not exclude other elements or steps. Unless specifically defined herein, all terms used herein have the same meaning as they would to one skilled in the art of the present invention.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure. Practitioners are particularly directed to Sambrook et al., Molecular Cloning: A Laboratory Manual, 4th ed., Cold Spring Harbor Press, Plainsview, New York (2012); and Ausubel et al., current Protocols in Molecular Biology (Supplement 100), John Wiley & Sons, New York (2012), for definitions and terms of the art. The definitions provided herein should not be construed to have a scope less than understood by a person of ordinary skill in the art.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). For example, if it is stated that an oligonucleotide of the present disclosure reduces expression of Syngr3 protein in a cell following administration of an oligonucleotide of the present disclosure by at least about 60%, it is implied that the Syngr3 expression levels are reduced by a range of 50% to 70%.

The terms “reverse complement”, “reverse complementary” and “reverse complementarity” as used herein are interchangeable with the terms “complement”, “complementary” and “complementarity”.

The terms “identical” or percent “identity” in the context of two or more nucleic acids refer to two or more sequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

The term “percent sequence identity” or “percent identity” between two polynucleotide or polypeptide sequences refers to the number of identical matched positions shared by the sequences over a comparison window, taking into account additions or deletions (i.e. gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

One such non-limiting example of a sequence alignment algorithm is the algorithm described in Karlin et al., 1990, Proc. Natl. Acad. Sci., 87:2264-2268, as modified in Karlin et al., 1993, Proc. Natl. Acad. Sci., 90:5873-5877, and incorporated into the NBLAST and XBLAST programs (Altschul et al., 1991, Nucleic Acids Res., 25:3389-3402). In certain aspects, Gapped BLAST can be used as described in Altschul et al., 1997, Nucleic Acids Res. 25:3389-3402. BLAST-2, WU-BLAST-2 (Altschul et al., 1996, Methods in Enzymology, 266:460-480), ALIGN, ALIGN-2 (Genentech, South San Francisco, California) or Megalign (DNASTAR) are additional publicly available software programs that can be used to align sequences. In certain aspects, the percent identity between two nucleotide sequences is determined using the GAP program in the GCG software package (e.g., using a NWSgapdna.CMP matrix and a gap weight of 40, 50, 60, 70, or 90 and a length weight of 1, 2, 3, 4, 5, or 6). In certain alternative aspects, the GAP program in the GCG software package, which incorporates the algorithm of Needleman and Wunsch (J. Mol. Biol. (48):444-453 (1970)) can be used to determine the percent identity between two amino acid sequences (e.g., using either a BLOSUM 62 matrix or a PAM250 matrix, and a gap weight of 16, 14, 12, 10, 8, 6, or 4 and a length weight of 1, 2, 3, 4, 5). Alternatively, in certain aspects, the percent identity between nucleotide or amino acid sequences is determined using the algorithm of Myers and Miller (CABIOS, 4:11-17 (1989)). For example, the percent identity can be determined using the ALIGN program (version 2.0) and using a PAM120 with residue table, a gap length penalty of 12 and a gap penalty of 4. One skilled in the art can determine appropriate parameters for maximal alignment by particular alignment software. In certain aspects, the default parameters of the alignment software are used.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI (European Bioinformatics Institute). In certain aspects, the percentage identity “X” of a first nucleotide sequence to a second nucleotide sequence is calculated as 100×(Y/Z), where Y is the number of amino acid residues scored as identical matches in the alignment of the first and second sequences (as aligned by visual inspection or a particular sequence alignment program) and Z is the total number of residues in the second sequence. If the length of a first sequence is longer than the second sequence, the percent identity of the first sequence to the second sequence will be higher than the percent identity of the second sequence to the first sequence. Different regions within a single polynucleotide target sequence that align with a polynucleotide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

In determining the degree of “complementarity” between oligomers of the disclosure (or regions thereof) and the target region, such as those disclosed herein, the degree of “complementarity” (also, “homology” or “identity”) is expressed as the percentage identity (or percentage homology) between the sequence of the oligomer (or region thereof) and the sequence of the target region (or the reverse complement of the target region) that best aligns therewith. The percentage is calculated by counting the number of aligned bases that are identical between the two sequences, dividing by the total number of contiguous monomers in the oligomer, and multiplying by 100. In such a comparison, if gaps exist, it is preferable that such gaps are merely mismatches rather than areas where the number of monomers within the gap differs between the oligomer of the disclosure and the target region.

As used in the present disclosure, the terms “nucleic acid molecule of the invention” and “oligonucleotide of the present disclosure” and grammatical variants thereof are used interchangeably. The term “defined by SEQ ID No. X” as used herein refers to a biological sequence consisting of the sequence of amino acids or nucleotides given in the SEQ ID No. X.

“Specific to synaptogyrin-3” as used herein is referring to the fact that the nucleic acid molecule or oligonucleotide of the invention is acting at the level of synaptogyrin-3 and not at the level of another transcript. Specificity can be ascertained by e.g. determining the expression level of closely related RNA sequences.

### Antisense Technology

The concept of designing oligonucleotides to bind to specific sequences in target RNAs via Watson-Crick hydrogen bonding and the term antisense were introduced already in the late 1970s. The term “antisense” included at that time oligonucleotides of any structure, with any chemical modification and designed to work through any post-RNA hybridization mechanism. However, nowadays and in practice the term “antisense” is used to describe single stranded antisense oligonucleotides (ss ASOs) designed to hybridize to RNAs, while RNAi (RNA interference) mediating agents such as “siRNAs” have come to mean double stranded oligonucleotides designed to activate Argonaute2 (Ago2). Thus, in some aspects, an oligonucleotide of the present disclosure comprises an antisense oligonucleotide (ASO), e.g., an unconjugated or conjugated ASO. in some aspects, an oligonucleotide of the present disclosure comprises a siRNA, e.g., an unconjugated or conjugated siRNA.

### Antisense Oligonucleotides

In some aspects, the oligonucleotide of the present disclosure is an ASO. Antisense oligonucleotides or ASOs are small (generally, ^{˜}18-30 nucleotides or shorter, e.g. 12 to 20 nucleotides), synthetic, single-stranded nucleic acid polymers of diverse chemistries, which can be employed to modulate gene expression via various mechanisms. ASOs can be subdivided into two major categories: RNase H competent and steric block ASOs. In some aspects, the oligonucleotide of the present disclosure is RNase H competent. In some aspects, the oligonucleotide of the present disclosure is a steric block ASO.

RNases H are a family of endonucleases that hydrolyze RNA residues in various nucleic acids, such as RNA-DNA-like duplexes. In human cells, there are two canonical RNase H enzymes: RNA H1 and H2. RNase H1 is present in the nucleus, cytoplasm and in mitochondria. The main function of RNase H1 is the clearance of R-Loops, particularly in GC rich genomic regions. The endogenous RNase H enzyme RNaseH1 also recognizes RNA-DNA heteroduplex substrates that are formed when DNA-based oligonucleotides are taken up by the cell and bind to complementary mRNA transcripts and subsequently catalyses the degradation of targeted RNA. Cleavage at the site of ASO binding results in destruction of the target RNA, thereby silencing target gene expression. To create a heteroduplex substrate for RNase H1, an ASO must contain at least 5 contiguous deoxynucleotide units, with optimal enzyme activity achieved with 8-10 contiguous deoxynucleotides. This approach has been widely used as a means of downregulating disease-causing or disease-modifying genes (Roberts et al 2020 Nature Reviews 19:673-694). Gapmers are non-limiting examples of RNase H-competent antisense oligonucleotides. They follow the ‘gapmer’ pattern, whereby a central DNA-based ‘gap’ (typically 8 to 10 deoxynucleotides in length) is surrounded by RNA-based (but chemically modified) flanking regions that promote target binding. In some aspects, the oligonucleotide of the present disclosure is a gapmer.

ASOs can also modulate gene expression by steric hindrance or occupancy-only mechanisms. Steric block oligonucleotides are designed to bind to target transcripts with high affinity but do not induce target transcript degradation as they lack RNase H competence. Such oligonucleotides therefore comprise either nucleotides that do not form RNase H substrates when paired with RNA or a mixture of nucleotide chemistries such that runs of consecutive DNA-like bases are avoided. Steric block oligonucleotides can mask specific sequences within a target transcript and thereby interfere with transcript RNA-RNA and/or RNA-protein interactions. The most widely used application of steric block ASOs is in the modulation of alternative splicing in order to selectively exclude or retain a specific exon(s) in order to disrupt the translation of the target gene. ASOs can also be designed to interfere with maturation and stability of the RNA transcript or to block its interaction with the translation apparatus.

In case the ASO can enter the nucleus, mRNA maturation can be modulated by inhibition of 5′ cap formation, inhibition of mRNA splicing or activation of RNaseH (Chan et al 2006 Clin Exp Pharmacol Physiol 33:533-540; this reference also describes some of the software available for assisting in design of ASOs).

### RNAi Using Duplex Silencers

In some aspects, the oligonucleotide of the present disclosure is the antisense portion of an RNAi duplex (double stranded RNA). RNA interference (RNAi) is a mechanism by which double-stranded RNA triggers the loss of a homologous RNA molecule. Short interfering RNA (siRNA) molecules are the effector molecules of RNAi and classically consist of a duplex of RNA molecules with a length of 21 nucleotides, i.e. 19 complementary bases and 2 terminal 3′ overhangs. One of the strands of the siRNA (the guide or antisense strand) is complementary to a target transcript, whereas the other strand is designated the passenger or sense strand. siRNAs act to guide the Argonaute2 protein (AGO2), as part of the RNA-induced silencing complex (RISC), to complementary target transcripts. Complete complementarity between the siRNA and the target transcript results in cleavage of the target opposite position of the guide strand, catalysed by AGO2, leading to gene silencing.

In an siRNA, the sense strand meets the formal definition of a drug delivery device: it is non-covalently bound, enhances the stability of the antisense strand and must be removed by the Ago2 loading complex before the pharmacophore, the antisense strand, is active.

Numerous variations of the archetypal siRNA design have been developed in terms of reduced passenger strand activity and/or improved potency. These include Dicer substrate siRNAs, small internally segmented siRNAs, self-delivering siRNAs (asymmetric and hydrophobic), single-stranded siRNAs and divalent siRNAs.

In some aspects, the oligonucleotide of the present disclosure is the antisense portion of a shRNA. Short hairpin RNAs (shRNAs) are artificial RNA molecules that are transcribed as a single stranded RNA but because of internal complementarity form a loop or hairpin-like structure. The hairpin is subsequently processed to an siRNA and also leads to the degradation of mRNAs in a sequence-specific manner dependent upon complementary binding of the target mRNA. shRNAs are slightly larger than siRNA molecules and, unlike siRNAs, are produced inside the cell in the nucleus.

Other non-limiting examples of RNAi-mediated duplex silencers are miRNAs and di-siRNAs. microRNAs (miRNAs) are endogenous non-coding RNA molecules that trigger RNAi and that have been implicated in a multitude of physiological and pathophysiological processes. miRNA hairpins embedded within long primary miRNA transcripts are sequentially processed by two RNase III family enzymes, DICER1 (Dicer) and DROSHA, which liberate the hairpin and then cleave the loop sequence, respectively. The resulting duplex RNA is analogous to an siRNA and is then loaded into an Argonaute protein (for example, AGO2) while one strand is discarded to generate the mature, single-stranded miRNA species. As with siRNAs, miRNAs guide RISC to target sequences where they initiate gene silencing. In contrast with siRNAs, miRNAs typically bind with partial complementarity and induce silencing via slicer-independent mechanisms.

In some aspects, the oligonucleotide of the present disclosure is a di-siRNA. Divalent siRNAs (di-siRNAs) are recently developed RNA silencing agent alternatives and have been shown to support a potent, sustained gene silencing in the central nervous system of mice and non-human primates following a single injection into the cerebrospinal fluid (Alterman et al 2019 Nature Biotech 37, 884-894). Di-siRNAs are composed of two fully chemically modified, phosphorothioate-containing siRNAs connected by a linker.

### Chemical Modifications

In some aspects, the oligonucleotides of the present disclosure comprise non-naturally occurring nucleotide analogues, e.g., nucleotides which have modified sugar moieties, such as bicyclic nucleotides or 2′ modified nucleotides, such as 2′ substituted nucleotides. An essential step in the evolution of the antisense technology was the creation, innovation and evaluation of the medicinal chemistry of oligonucleotides. The goals were to enhance the affinity for the target sequence (thereby increasing potency), assure effective distribution to peripheral tissues, enhance the duration of action by increasing resistance to degradation by nucleases, improve pharmacokinetic characteristics, reduce the class generic (chemically based) toxicities of the chemical classes widely used for therapeutics, and create designs that support multiple post-binding mechanisms, thereby broadening the utility of the technology.

Within the antisense field a broad effort was initiated to modify essentially every position in a dinucleotide except those required for Watson-Crick base pairing. Thousands of analogues have been synthesized and evaluated so far, and novel analogues continue to be investigated. Three major classes of modifications can be distinguished: modifications of the internucleotide linkage, alterations of the ribose sugar and bioconjugations with for example GalNAc.

### Phosphorothioates

In some aspects, the oligonucleotides of the present disclosure comprise one or more non-cleavable internucleotide linkages, e.g., phosphorothioate linkages. The phosphodiester backbone of unmodified DNA and RNA oligonucleotides is highly susceptible to degradation by nucleases in vivo. So, to develop oligonucleotides for therapeutic applications, it was necessary to identify backbone modifications that reduce their susceptibility to nuclease degradation while not compromising other key characteristics such as RNase H1 activation and RNA binding too much.

In phosphorothioate (PS) linkages, a non-bridging oxygen in the phosphate group is substituted by sulfur. The PS moiety provides significant protection against nucleases. Importantly, because of the impact of the greater size of sulfur compared with oxygen, the negative charge of the PS moiety at physiological pH is more widely distributed than in a phosphodiester (PO) moiety. This increases the lipophilicity of oligonucleotides that contain PS moieties, facilitating binding to proteins and thereby preventing rapid excretion of the oligonucleotides by the kidney and facilitating uptake of oligonucleotides into cells and tissues. The PS moiety is the most widely used backbone modification in ASOs and siRNAs.

### Ribose Sugar Modifications

In some aspects, the oligonucleotides of the present disclosure comprise non-naturally occurring nucleotide analogues, e.g., nucleotides which have modified sugar moieties, such as bicyclic nucleotides or 2′ modified nucleotides, such as 2′ substituted nucleotides Oligonucleotides are frequently modified at the ribose sugar, primarily with the aim of improving properties such as affinity and/or nuclease resistance. Such modifications include those where the ribose ring structure is modified (e.g. locked nucleic acids or LNAs), where the sugar moiety is replaced by a non-sugar moiety (e.g. peptide nucleic acids or PNAs) or where the substituent groups on the ribose ring are altered to groups other than the hydrogen or 2′-OH group naturally found in DNA and RNA nucleosides.

Non-limiting examples of ring structure modifications are HNAs where ribose ring is replaced with a hexose ring, an UNA (unlocked nucleic acid) where an unlinked ribose ring lacks a bond between the C2 and C3 carbons or a Locked Nucleic Acid (LNA) where the C2′ and C4′ of the ribose sugar ring are linked by a methylene bridge (also referred to as a “2′-4′ bridge”), which restricts or locks the conformation of the ribose ring. The locking of the conformation of the ribose (also referred to as Bridged Nucleic Acids or BNAs) is associated with an enhanced affinity of hybridization (duplex stabilization) when the LNA is incorporated into an oligonucleotide for a complementary RNA or DNA molecule. Non limiting examples of LNA nucleosides are beta-D-oxy-LNA, 6′-methyl-beta-D-oxy LNA such as (S)-6′-methyl-beta-D-oxy-LNA (ScET) and 2′-O,4′-C-ethylene-bridged nucleic acid (ENA) or those disclosed in WO 1999/014226, WO 2000/66604, WO 1998/039352, WO 2004/046160, WO 2000/047599, WO 2007/134181, WO 2010/077578, WO 2010/036698, WO 2007/090071, WO 2009/006478, WO 2011/156202, WO 2008/154401, WO 2009/067647, WO 2008/150729.

Since BNA modifications enhance both nuclease stability and the affinity of the oligonucleotide for target RNA, they have been incorporated into the flanking regions of gapmers to improve target binding. As such, cEt-flanking 3-10-3 gapmers are more efficacious than the MOE 5-10-5 equivalents. Importantly, BNAs are excluded from the DNA gap region because they are not compatible with RNase H-mediated cleavage. LNA modifications have also been utilized in steric block ASOs, such as miRNA inhibitors.

Non-limiting examples of 2′ substituted modified nucleosides are 2′-O-alkyl-RNA, 2′-O-methyl-RNA (2′-OMe), 2′-alkoxy-RNA, 2′-O-methoxyethyl-RNA (MOE), 2′-amino-DNA, 2′-Fluoro-RNA (2′-F), and 2′-F-ANA nucleoside. These modifications increase oligonucleotide nuclease resistance by replacing the nucleophilic 2′-hydroxyl group of unmodified RNA, leading to improved stability in plasma, increased tissue half-lives and consequently prolonged drug effects. These modifications also enhance the binding affinity of the oligonucleotide for complementary RNA and some 2′ modifications reduce pro-inflammatory effects. The 2′-ribose modifications are not compatible with RNase H activity, meaning they are typically used for steric block oligonucleotides, or for the flanking sequences in gapmer ASOs. Although most effort was done in modifying the 2′ position, substituents can be introduced at the 3′, 4′ or 5′ positions as well.

The present disclosure provides oligonucleotides comprising or consisting of a simple sequence of natural occurring nucleotides—preferably 2′-deoxynucleotides (referred here generally as “DNA”), but also possibly ribonucleotides (referred here generally as “RNA”), or a combination of such naturally occurring nucleotides and one or more non-naturally occurring nucleotides, i.e., “nucleotide analogues”, such as nucleotides having the ribose sugar modifications disclosed above.

In some aspects, the oligonucleotide of the present disclosure comprises at least two nucleotide analogues. In some aspects, the oligonucleotide of the present disclosure comprises from 3, 4, 5, 6, 7, or 8 nucleotide analogues, e.g. 6 or 7 nucleotide analogues. In some aspects, all the nucleotide analogues are the same. In some aspects, some nucleotide analogs are different. In some aspects, all the nucleotides in the oligonucleotide of the present disclosure are nucleotide analogues. In some aspects, when all the nucleotides in the oligonucleotide of the present disclosure are nucleotide analogues, all the nucleotide analogues are the same. In some aspects, when all the nucleotides in the oligonucleotide of the present disclosure are nucleotide analogues, some of the nucleotide analogues are different.

### Conjugation

In some aspects, the oligonucleotide of the present disclosure is a conjugate, e.g., a GalNAc conjugate. The delivery potential of ASOs and siRNAs can be enhanced through direct covalent conjugation of various moieties that promote intracellular uptake, target the drug to specific cells/tissues or reduce clearance from the circulation. Non-limiting examples are lipids, peptides, aptamers, antibodies and sugars. Bioconjugates constitute distinct, homogeneous, single-component molecular entities with precise stoichiometry, meaning that high-scale synthesis is relatively simple and their pharmacokinetic properties are well defined. Furthermore, bioconjugates are typically of small size meaning that they generally exhibit favourable biodistribution profiles. For example, conjugating ASOs or siRNAs to the sugar moiety GalNAc results in more productive delivery to hepatocytes without a meaningful shift in distribution to other tissues and results in 15-30 fold increases in potency for RNA targets in those cells. ASOs and siRNAs can also be loaded to exosomes. Exosomes are heterogeneous, lipid bilayer-encapsulated vesicles approximately 100 nm in diameter that are generated as a result of the inward budding of the multivesicular bodies. Exosomes are thought to be released into the extracellular space by all cells, where they facilitate intercellular communication via the transfer of their complex macromolecular cargoes. Exosomes present numerous favourable properties in terms of oligonucleotide drug delivery of which crossing biological membranes, such as the blood-brain-barrier (BBB) is highly relevant for treatments of CNS disorders.

### Nucleic Acid Molecules Inhibiting the Expression of Synaptogyrin-3

In a first aspect, the application discloses nucleic acid molecules that comprise a sequence complementary to a region of Synaptogyrin-3 as depicted in SEQ ID No. 1 or SEQ ID No. 3 or allelic variants thereof.

SEQ ID No. 1 represent the DNA sequence of the mouse synaptogyrin-3 gene (Gene ID: 20974). It is 4964 bp long, comprises 4 exons and encodes the mouse synaptogyrin-3 protein of which the amino acid sequence is depicted in SEQ ID No. 2.

The mouse synaptogyrin-3 gene shares 80% homology with the human synaptogyrin-3 gene (Gene ID: 9143) as depicted in SEQ ID No. 3, encoding the human SYNGR3 protein of which the amino acid sequence is depicted in SEQ ID No. 4.

The nucleic acid molecules or the oligonucleotides referred to in the invention are generally oligonucleotides of between 10 and 50 nucleotides in length and composed of 1 or 2 oligonucleotides.

In some embodiments, the nucleic acid molecules of the invention comprise or consist of 8 to 70 nucleotides in length, 10 to 60 nucleotides in length, 12 to 50 nucleotides in length, 8 to 40 nucleotides in length, or from 9 to 35, from 10 to 30, from 11 to 22, from 12 to 20, from 13 to 18 or from 14 to 16 contiguous nucleotides in length. In some aspects, the nucleic acid molecule of the invention is 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 47, 48, 49, 50, 51, 52, 53, 54, 55, 56, 57, 58, 59, or 60 nucleotides in length.

“Nucleotides” as used herein refer to the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which are absent in nucleosides). A nucleotide without a phosphate group is called a “nucleoside” and is thus a compound comprising a nucleobase moiety and a sugar moiety. As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Naturally occurring nucleobases of RNA or DNA comprise the purine bases adenine (A) and guanine (G), and the pyrimidine bases thymine (T), cytosine (C) and uracil (U).

The term “contiguous nucleotides” or “contiguous nucleotide sequence” as used herein refers to the uninterrupted region of the oligonucleotide which is complementary to the target nucleic acid. “Contiguous” as used herein means next or together in sequence, hence the contiguous nucleotides are linked nucleotides (i.e. no additional nucleosides are present between those that are linked). The target nucleic acid of the invention is Synaptogyrin-3, more particularly human Synaptogyrin-3. “Synaptogyrin3”, “synaptogyrin-3”, “Syngr3”, “Syngr-3”, “SYNGR3” or “SYNGR-3” are interchangeably used and refer herein to synaptogyrin 3 transcript if not otherwise specified. The mouse nucleic acid sequence of Synaptygyrin-3 (mSyngr-3) is depicted in SEQ ID No. 1 and the human nucleic acid sequence of Synaptogyrin-3 (hSyngr-3) is depicted in SEQ ID No. 3, however also within the scope of the invention are nucleic acid sequence variants of Synaptogyin-3 as may exist due to allelic variation. Such variations are defined herein as “allelic variants of SEQ ID No. 1 and/or 3”. The term “allelic variants” refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

The term “oligomer” or “oligonucleotide” in the context of the present disclosure are used interchangeably, and refer to a molecule formed by covalent linkage of two or more nucleotides. Herein, a single nucleotide (unit) can also be referred to as a monomer or unit.

In some aspects, the present disclosure provides a derivative of an oligonucleotide of the present disclosure which is a conjugate, e.g., a GalNAc conjugate. The term “derivative” as used herein refers to a chemical compound related structurally to a compound disclosed herein (e.g., an oligonucleotide of the present disclosure), e.g., having the same carbon skeleton, but chemically modified to introduce, e.g., a side chain or group, in one or more positions, and wherein the derivative possesses a biological activity (e.g., the capacity to reduce Syngr3 expression) that is substantially similar to a biological activity of the entity or molecule it is a derivative.

The term, “complementary” means that two sequences are complementary when the sequence of one can bind to the sequence of the other in an anti-parallel sense wherein the 3′-end of each sequence binds to the 5′-end of the other sequence and each A, T(U), G, and C of one sequence is then aligned with a T(U), A, C, and G, respectively, of the other sequence. Normally, the complementary sequence of the oligonucleotide has at least 90%, preferably 95%, most preferably 100%, complementarity to a defined sequence.

In determining the degree of “complementarity” between oligomers of the disclosure (or regions thereof) and the target region, such as those disclosed herein, the degree of “complementarity” (also, “homology” or “identity”) is expressed as the percentage identity (or percentage homology) between the sequence of the oligomer (or region thereof) and the sequence of the target region (or the reverse complement of the target region) that best aligns therewith. The percentage is calculated by counting the number of aligned bases that are identical between the two sequences, dividing by the total number of contiguous monomers in the oligomer, and multiplying by 100. In such a comparison, if gaps exist, it is preferable that such gaps are merely mismatches rather than areas where the number of monomers within the gap differs between the oligomer of the disclosure and the target region.

In some embodiments, the nucleic acid molecule described above or the contiguous nucleotide sequence thereof comprises or consists of 24 or less nucleotides, such as 22 or less nucleotides, such as 20 or less nucleotides, such as 18 or less nucleotides, such as 14, 15, 16 or 17 nucleotides. It is to be understood that any range given herein includes the range endpoints. Accordingly, if a nucleic acid molecule is said to include from 10 to 30 nucleotides, both 10 and 30 nucleotides are included. In some embodiments, the contiguous nucleotide sequence comprises or consists of 8, 9, 10, 11, 12, 13, 14, 15, 16, 17, 18, 19, 20, 21, 22, 23, 24, 25, 26, 27, 28, 29 or 30 contiguous nucleotides in length. In some aspects, the nucleic acid molecule of the invention is 14 nucleotides in length. In some aspects, the nucleic acid molecule of the invention is 15 nucleotides in length. In some aspects, the nucleic acid molecule of the invention is 16 nucleotides in length. In some aspects, the nucleic acid molecule of the invention is 17 nucleotides in length. In some aspects, the nucleic acid molecule of the invention is 18 nucleotides in length. In some aspects, the nucleic acid molecule of the invention is 19 nucleotides in length. In some aspects, the nucleic acid molecule of the invention is 20 nucleotides in length.

The nucleic acid molecule(s) is typically for modulating the expression of Synaptogyrin-3 as target nucleic acid in a mammal. In some embodiments the nucleic acid molecule(s), such as siRNAs, shRNAs or antisense oligonucleotides, is typically for inhibiting the expression of a target nucleic acid.

In another embodiment, an oligonucleotide is provided of 10 to 50 nucleotides in length or of 10 to 40 or of 10 to 30 nucleotides in length, comprising a contiguous nucleotide sequence of at least 10 contiguous nucleotides in length which are at least 90% complementary to a region of Synaptogyrin-3 as depicted in SEQ ID No. 1 or allelic variants thereof, wherein the region is selected from the group consisting of position 234-249; 237-253; 1144-1159; 1205-1220; 1319-1334; 2271-2287; 2295-2311; 2297-2316; 2298-2314; 3387-3402; 3470-3486; 3570-3586; 3617-3633; 4561-4577; and 4683-4699 of SEQ ID No. 1. Said regions are referred herein as “the regions of SEQ ID No. 1 of current application”. This is equivalent as saying that an oligonucleotide is provided of 10 to 50, or of 10 to 40 or of 10 to 30 nucleotides in length, comprising a contiguous nucleotide sequence of at least 10 contiguous nucleotides in length which are at least 90% complementary to a region of Synaptogyrin-3 selected from TCCCGTGAGCTTTGCG, CGTGAGCTTTGCGCGGC, AGAATTGTGGAATGAT, AATGACTGTGGTTTGC, ACTACTCACGGGAATC, TGTCAACGAGGGCTACG, TCAACGAGGGCTACGTGAAC, CAACGAGGGCTACGTGA, CCAATCAGTGGCAACG, TTCTCCATCCTCAGCTG, CGCTCACCGTGAAGGCC, TCTCTCTTTGCCACAGA, TAGTCTATCTTCTATCC or GTCTCATTCTTCCAGAC, or alternatively phrased selected from SEQ ID No. 38-51.

In another embodiment, an oligonucleotide is provided of 10 to 50, 10 to 40 or 10 to 30 nucleotides in length, comprising a contiguous nucleotide sequence of at least 10 contiguous nucleotides in length which are at least 90% complementary to region 234-253 of SEQ ID No. 1 (i.e. TCCCGTGAGCTTTGCGCGGC or SEQ ID No. 52), region 235-253 of SEQ ID No. 1 (i.e. CCCGTGAGCTTTGCGCGGC or SEQ ID No. 53) or to region 2295-2316 of SEQ ID No. 1 (i.e. TGTCAACGAGGGCTACGTGAAC or SEQ ID No. 54).

In a particular embodiment, the contiguous nucleotide sequence is at least 80%, at least 81%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% complementary to the Syngr-3 region as disclosed above and herein. In a particular embodiment, the contiguous nucleotide sequence is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 100% complementary to the Syngr-3 region as disclosed above and herein. In another particular embodiment, the contiguous nucleotide sequence is complementary to an equal length portion of SEQ ID No. 1.

The mouse-human conserved regions within the Synaptogyrin-3 transcript that are significantly more accessible for antisense molecules and therefore are preferred regions for designing oligonucleotides for reducing the expression of Syngr-3, were further fine-tuned in a human Syngr-3 context. By transcript-walking the borders of the identified regions were determined. The threshold for a region suitable for oligonucleotide design was set at the level of the oligonucleotide, i.e. the oligonucleotide should be able to reduce the human Syngr-3 transcript with at least 45% with respect to a reference system (e.g., baseline levels in an individual or population of individuals, or below a pre-determined threshold value). Nevertheless, therapeutic effects may be obtained at levels of inhibition of the expression of the human Syngr-3 above or below 45%. For example, the level of inhibition can be about 20%, about 25%, about 30%, about 35%, about 40%, about 45%, about 55%, about 60%, about 65%, about 70%, about 75%, about 80%, about 85%, about 90%, about 95%, or about 100%. In some, the level of inhibition can be at least about 20%, at least about 25%, at least about 30%, at least about 35%, at least about 40%, at least about 45%, at least about 55%, at least about 60%, at least about 65%, at least about 70%, at least about 75%, at least about 80%, at least about 85%, at least about 90%, at least about 95%, or about 100%.

The application thus provides regions within the human Syngr-3 gene for designing oligonucleotides that reduce the expression of hSYNGR3 with at least 45%, or other levels as disclose above, compared to a control situation, e.g. where no antisense molecule was administer or where a scrambled antisense molecule was added as negative control. More particularly, said regions are selected from the list consisting of the regions between position 470 and 511, between position 2344 and 2368 and between position 3170 and 3236 of SEQ ID No. 3, wherein the endpoints are included in the regions. Even more particularly, said region is region 480-511, 485-511, 490-511, 492-511, 2346-2366, 3180-3236, 3185-3236 or 3188-3236 of SEQ ID No. 3.

As used herein, the term “reducing”, e.g., reducing the expression of hSYNGR3 gene transcript and/or hSYNGR3 protein level or hSYNGR3 activity refers to the oligonucleotide of the present disclosure (e.g., an ASO or siRNA) disclosed herein reducing the expression of the hSYNGR3 gene transcript and/or hSYNGR3 protein level and/or activity in a cell, a tissue, or a subject.

In some aspects, the term “reducing” refer to complete inhibition (100% inhibition or non-detectable level) of hSYNGR3 gene transcript or hSYNGR3 protein level and/or activity. In other aspects, the term “reducing” refers, e.g., to at least about 45%, at least about 50%, at least about 55%, at least about 60%, at least about 65%, at least about 70%, at least about 75%, at least about 80%, at least about 85%, at least 90%, at least 95% or at least 99% inhibition of hSYNGR3 gene transcript and/or hSYNGR3 protein expression and/or activity in a cell, a tissue, or a subject.

The terms “individual”, “subject”, “host”, and “patient”, are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

In some aspects, the present disclosure provides a target region within the human Syngr-3 gene that can be used for designing oligonucleotides or antisense molecules, wherein the region is depicted in SEQ ID No. 69 consisting of CCGCGCAGGGGCCGCCCTGGACCCCGTGAGCTTTGCGCGGCG,
more particularly in SEQ ID No. 70 and consisting of GCCGCCCTGGACCCCGTGAGCTTTGCGCGGCG,
more particularly in SEQ ID No. 71 and consisting of CCTGGACCCCGTGAGCTTTGCGCGGCG,
more particularly in SEQ ID No. 72 and consisting of ACCCCGTGAGCTTTGCGCGGCG,
more particularly in SEQ ID No. 73 and consisting of CCCGTGAGCTTTGCGCGGCG.

Thus, the present disclosure provides a method for designing or manufacturing an oligonucleotide of the present disclosure (e.g., an ASO or a siRNA) capable of inhibiting a hSYNGR3 gene transcript and/or hSYNGR3 protein expression and/or activity in a cell, a tissue, or a subject, wherein the oligonucleotide of the present disclosure is complementary (partially or fully complementary) to a nucleotide sequence set for in SEQ ID No. 69, 70, 71, 72, or 73, or a subsequence thereof. In some aspects, the complementary sequence of the oligonucleotide of the present disclosure corresponds to a subsequence of a nucleotide sequence set for in SEQ ID No. 69, 70, 71, 72, or 73. In some aspects, the complementary sequence of the oligonucleotide of the present disclosure partially overlaps of a nucleotide sequence set for in SEQ ID No. 69, 70, 71, 72, or 73.

Also a target region within the human Syngr-3 gene is provided for designing antisense molecules, wherein the region is depicted in SEQ ID No. 74 consisting of CGTCAACGAGGGCTACGTGAACACC, more particularly in SEQ ID No. 75 and consisting of TCAACGAGGGCTACGTGAACA.

Thus, the present disclosure provides a method for designing or manufacturing an oligonucleotide of the present disclosure (e.g., an ASO or a siRNA) capable of inhibiting a hSYNGR3 gene transcript and/or hSYNGR3 protein expression and/or activity in a cell, a tissue, or a subject, wherein the oligonucleotide of the present disclosure is complementary (partially or fully complementary) to a nucleotide sequence set for in SEQ ID No. 74 or 75, or a subsequence thereof. In some aspects, the complementary sequence of the oligonucleotide of the present disclosure corresponds to a subsequence of a nucleotide sequence set for in SEQ ID No. 74 or 75. In some aspects, the complementary sequence of the oligonucleotide of the present disclosure partially overlaps of a nucleotide sequence set for in SEQ ID No. 74 or 75.

Also a target region within the human Syngr-3 gene is provided for designing antisense molecules, wherein the region is depicted in SEQ ID No. 76 consisting of TCCCGGCTGACCCCGCTGACCCCGCCCCGCGCAGGTGGCGCTCACCGTGAAGGCCCTGCAGCGGTTC, more particularly in SEQ ID No. 77 and consisting of CCCCGCTGACCCCGCCCCGCGCAGGTGGCGCTCACCGTGAAGGCCCTGCAGCGGTTC,
even more particularly in SEQ ID No. 78 and consisting of CTGACCCCGCCCCGCGCAGGTGGCGCTCACCGTGAAGGCCCTGCAGCGGTTC
even more particularly in SEQ ID No. 79 and consisting of ACCCCGCCCCGCGCAGGTGGCGCTCACCGTGAAGGCCCTGCAGCGGTTC.

Thus, the present disclosure provides a method for designing or manufacturing an oligonucleotide of the present disclosure (e.g., an ASO or a siRNA) capable of inhibiting a hSYNGR3 gene transcript and/or hSYNGR3 protein expression and/or activity in a cell, a tissue, or a subject, wherein the oligonucleotide of the present disclosure is complementary (partially or fully complementary) to a nucleotide sequence set for in SEQ ID No. 76, 77, 79 or 79, or a subsequence thereof. In some aspects, the complementary sequence of the oligonucleotide of the present disclosure corresponds to a subsequence of a nucleotide sequence set for in SEQ ID No. 76, 77, 79 or 79. In some aspects, the complementary sequence of the oligonucleotide of the present disclosure partially overlaps of a nucleotide sequence set for in SEQ ID No. 76, 77, 79 or 79.

As used herein the term “manufacturing” refers to chemically synthesizing, e.g., using solid phase synthesis, an oligonucleotide of the present disclosure. In some aspects, manufacturing further comprises chemically attaching or conjugating a moiety such a delivery moiety (e.g., a GalNAc moiety), and/or a targeting moiety.

In another embodiment, an oligonucleotide is provided of 10 to 50 nucleotides in length or of 10 to 40 or of 10 to 30 nucleotides in length, comprising a contiguous nucleotide sequence of at least 10 contiguous nucleotides in length which are at least 90% complementary to a region of Synaptogyrin-3 as depicted in SEQ ID No. 3 or allelic variants thereof, wherein the region is selected from the group consisting of the regions between position 470 and 511, between position 2344 and 2368 and between position 3170 and 3236 of SEQ ID No. 3. In another embodiment, an oligonucleotide is provided of 10 to 50 nucleotides in length or of 10 to 40 or of 10 to 30 nucleotides in length, comprising a contiguous nucleotide sequence of at least 10 contiguous nucleotides in length which are at least at least 80%, at least 81%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% complementary to a region of Synaptogyrin-3 as depicted in SEQ ID No. 3 or allelic variants thereof, wherein the region is selected from the group consisting of the regions between position 470 and 511, between position 2344 and 2368 and between position 3170 and 3236 of SEQ ID No. 3. Even more particularly, said region is region 480-511, 485-511, 490-511, 492-511, 2346-2366, 3180-3236, 3185-3236 or 3188-3236 of SEQ ID No. 3. Said regions are referred herein as “the regions of SEQ ID No. 3 of current application” and said oligonucleotides are capable of reducing Synaptogyrin-3 expression, more particularly human Syngr-3.

In some aspects, an oligonucleotide is provided of 10 to 50, or of 10 to 40 or of 10 to 30 nucleotides in length, comprising a contiguous nucleotide sequence of at least 10, at least 11, at least 12, at least 13, at least 14, at least 15 or at least 16 contiguous nucleotides in length which are at least 90% complementary to a region of Synaptogyrin-3 selected from CCGCGCAGGGGCCGCCCTGGACCCCGTGAGCTTTGCGCGGCG, GCCGCCCTGGACCCCGTGAGCTTTGCGCGGCG, CCTGGACCCCGTGAGCTTTGCGCGGCG, ACCCCGTGAGCTTTGCGCGGCG, CCCGTGAGCTTTGCGCGGCG, CGTCAACGAGGGCTACGTGAACACC, TCAACGAGGGCTACGTGAACA, TCCCGGCTGACCCCGCTGACCCCGCCCCGCGCAGGTGGCGCTCACCGTGAAGGCCCTGCAGCGGTTC, CCCCGCTGACCCCGCCCCGCGCAGGTGGCGCTCACCGTGAAGGCCCTGCAGCGGTTC, CTGACCCCGCCCCGCGCAGGTGGCGCTCACCGTGAAGGCCCTGCAGCGGTTC, ACCCCGCCCCGCGCAGGTGGCGCTCACCGTGAAGGCCCTGCAGCGGTTC, or alternatively phrased selected from SEQ ID No. 69-79.

In a particular embodiment, the contiguous nucleotide sequence is at least 90%, 91%, 92%, 93%, 94%, 95%, 96%, 97%, 98%, 99% or 100% complementary to the SYNGR3 region as disclosed above and herein. In another particular embodiment, the contiguous nucleotide sequence is complementary to an equal length portion of SEQ ID No. 3.

In a particular embodiment, said oligonucleotide is provided wherein the contiguous nucleotide sequence is at least 10, 12, 14 or 16 nucleotides in length. In a more particular embodiment, said contiguous nucleotide sequence is 100% complementary to one of the regions of SEQ ID No. 1 or 3 of current application or allelic variants thereof. In a most particular embodiment, the 10 to 50, 10 to 40 or the 10 to 30 nucleotides of which the oligonucleotides according to the invention are comprised, are linked nucleotides. In another particular embodiment, all oligonucleotides herein described are provided for reducing the expression of Synaptogyrin-3, more particularly of mice and/or human Synaptogyrin-3.

In another embodiment, the oligonucleotide of the invention comprises a contiguous nucleotide sequence which is complementary to the target nucleic acid or target sequence, and may, in some embodiments further comprise one or more additional nucleotides, such as 1-30, such as 1-20, such as 1-10, such as 1, 2, 3, 4 or 5 further nucleotides in addition to the contiguous nucleotide sequence. In some embodiments the additional nucleotides are complementary to the contiguous nucleotide sequence and are capable of forming a stem loop (hairpin) structure by hybridizing to the contiguous nucleotide sequence. In some embodiments the additional nucleotides are 1 to 5 phosphodiester linked nucleotides. In some embodiments, all the nucleotides of the oligonucleotide form the contiguous nucleotide sequence.

In one embodiment, the nucleic acid molecule(s) or the oligonucleotide(s) of the invention is man-made and/or is chemically synthesized and/or is typically purified or isolated. Accordingly, the present disclosure provides a method of manufacturing the nucleic acid molecule(s) or the oligonucleotide(s) of the invention comprising chemically synthesizing the nucleic acid molecule(s) or the oligonucleotide(s) of the invention. In some aspects, the method comprises the conjugation of a delivery moiety, e.g., a GalNAc moiety.

In yet another embodiment, the nucleic acid molecule or oligonucleotide of the invention may be or comprise an antisense oligonucleotide (ASO) or may be another oligomeric nucleic acid molecule such as a CRISPR RNA, a siRNA, shRNA, an aptamer or a ribozyme. In a particular embodiment the nucleic acid molecule of the invention is a RNAi agent, more particularly a siRNA, a shRNA or a miRNA.

The term “RNAi agent” or “RNA interference (RNAi) molecule” refers to any molecule inhibiting RNA expression or translation via the RNA reducing silencing complex (RISC) in a cell's cytoplasm, where the RNAi molecule interacts with the catalytic RISC component argonaute. A small interfering RNA (siRNA) is typically a double-stranded RNA complex comprising a passenger (sense) and a guide (antisense) oligonucleotide (strand), which when administered to a cell, results in the incorporation of the guide (antisense) strand into the RISC complex (siRISC) resulting in the RISC associated inhibition of translation or degradation of complementary RNA target nucleic acids in the cell. The sense strand is also referred to as the passenger strand, and the antisense strand as the guide strand. A small hairpin RNA (shRNA) is a single nucleic acid molecule which forms a stem loop (hairpin) structure that is able to degrade mRNA via RISC. RNAi nucleic acid molecules may be synthesized chemically (typical for siRNA complexes) or by in vitro transcription, or expressed from a vector.

shRNA molecules are generally between 40 and 70 nucleotides in length, such as between 45 and 65 nucleotides in length, such as 50 and 60 nucleotides in length, and interacts with the endonuclease known as Dicer which is believed to processes dsRNA into 19-23 base pair short interfering RNAs with characteristic two base 3′ overhangs which are then incorporated into an RNA-induced silencing complex (RISC). Typically, the guide (antisense) strand of an siRNA (or antisense region of a shRNA) is 17-25 nucleotide in length, such as 19-23 nucleotides in length and complementary to the target nucleic acid or target sequence. In an siRNA complex, the guide (antisense) strand and passenger (sense) strand form a double stranded duplex, which may comprise 3′ terminal overhangs of e.g. 1-3 nucleotides (resembles the product produced by Dicer), or may be blunt ended (no overhang at one or both ends of the duplex). It will be recognized that RNAi may be mediated by longer dsRNA substrates which are processed into siRNAs within the cell (a process which is thought to involve the dsRNA endonuclease DICER), such as miRNAs.

In another particular embodiment the nucleic acid molecule of the invention is an antisense oligonucleotide, such as single stranded antisense oligonucleotide, such as a high affinity modified antisense oligonucleotide interacting with RNase H.

The term “antisense oligonucleotide” as used herein is defined as an oligonucleotide capable of modulating expression of a target gene by hybridizing to a target nucleic acid, in particular to a contiguous sequence on a target nucleic acid. The antisense oligonucleotides are not essentially double stranded and are therefore not siRNAs or shRNAs. Preferably, the antisense oligonucleotides of the present invention are single stranded. It is understood that single stranded oligonucleotides of the present invention can form hairpins or intermolecular duplex structures (duplex between two molecules of the same oligonucleotide), as long as the degree of intra or inter self-complementarity is less than 50% across of the full length of the oligonucleotide.

In a further particular embodiment, the single stranded antisense oligonucleotide of the invention does not contain RNA nucleosides, since this will decrease nuclease resistance. More particularly, the antisense oligonucleotide of the invention comprises one or more modified nucleosides or nucleotides, such as 2′ sugar modified nucleosides. Furthermore, it is advantageous that the nucleosides which are not modified are DNA nucleosides.

In one embodiment, the oligonucleotide, e.g. the therapeutic antisense oligonucleotide, shRNA or siRNA, of the invention comprises one or more internucleoside linkages modified from the natural phosphodiester, such one or more modified internucleoside linkages that is for example more resistant to nuclease attack. The term “modified internucleoside linkage” is defined as generally understood by the skilled person as linkages other than phosphodiester (PO) linkages, that covalently couples two nucleosides together. Increased resistance of the oligonucleotide towards nucleases compared to a phosphodiester linkage is particular advantage for therapeutic oligonucleotides. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester. In a particular embodiment, the modified internucleoside linkage is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate. The use of fully phosphorothioate modified oligonucleotides or contiguous nucleotide sequences is often used in antisense oligonucleotides, although in siRNAs partial phosphorothioate modifications may be preferred as fully phosphorothioate modifications have been reported to limit RNAi activity, particularly when used in the guide (antisense) strand. Phosphorothioate modifications may be incorporated into the 5′ and 3′ ends of an antisense strand of a siRNA without unduly limiting RNAi activity.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate. In particular embodiments, all the internucleoside linkages in the contiguous nucleotide sequence of the antisense oligonucleotide are phosphorothioate linkages.

In other embodiments, antisense oligonucleotide may comprise other internucleoside linkages (other than phosphodiester and phosphorothioate), for example alkyl phosphonate/methyl phosphonate internucleosides.

In some embodiments, the RNAi molecules of the invention comprise one or more phosphorothioate internucleoside linkages. In RNAi molecules phosphorothioate internucleoside linkages may reduce the nuclease cleavage in RICS and it is therefore advantageous that not all internucleoside linkages are modified. Phosphorothioate internucleoside linkages can advantageously be place in the 3′ and/or 5′ end of the RNAi molecule, in particular in part of the molecule that is not complementary to the target nucleic acid (e.g. the sense strand or passenger strand in an siRNA molecule). The region of the RNAi molecule that is complementary to the target nucleic acid (e.g. the antisense or guide strand in a siRNA molecule) may however also be modified in the first 2 to 3 internucleoside linkages in the 3′ and/or 5′ terminal.

In other embodiments, the oligonucleotides of the invention may be chemically modified by incorporating high affinity nucleosides such as 2′ sugar modified nucleosides, such as 2′-4′ bicyclic ribose modified nucleosides, including LNA and cET or 2′ substituted modifications like of 2′-O-alkyl-RNA, 2′-O-methyl-RNA, 2′-alkoxy-RNA, 2′-O-methoxyethyl-RNA (MOE), 2′-amino-DNA, 2′-fluoro-DNA, arabino nucleic acid (ANA), 2′-fluoro-ANA. See for example WO 2002/044321 which discloses 2′-O-Methyl modified siRNAs, WO2004083430 which discloses the use of LNA nucleosides in siRNA complexes, known as siLNAs, and WO2007107162 which discloses the use of discontinuous passenger strands in siRNA such as siLNA complexes.

In a particular embodiment, the oligonucleotide of the invention comprises a 2′ sugar modified nucleoside selected from the list consisting of 2′-O-methyl (2′-OMe), 2′-O-methoxyethyl (2′-MOE) and 2′-Fluoro (2′-F).

In other embodiments, the oligonucleotides of the invention may comprise one or more of the above described chemically modified sugar nucleosides and may comprise one or more of the above described phosphorothioate internucleoside linkages.

The skilled person is aware of how to design the oligonucleotides disclosed herein. siRNA and shRNA design programs are publicly available. Non-limiting examples are siDESIGN from ThermoScientific, siDirect (Naito et al), BLOCK-IT RNAi Designer from Invitrogen, siRNA Wizard from InvivoGen, shRNA design tool from Gene Link and shRNA design tool from transomic. Manufacturers of RNAi products also provide guidelines for designing siRNA/shRNA. siRNA sequences between 19-29 nucleotides (nt) are generally the most effective. Sequences longer than 30 nt can result in nonspecific silencing. Ideal sites to target include AA dinucleotides and the 19 nt 3′ of them in the target mRNA sequence. Typically, siRNAs with 3′ dUdU or dTdT dinucleotide overhangs are more effective. Other dinucleotide overhangs could maintain activity but GG overhangs should be avoided. Also to be avoided are siRNA designs with a 4-6 poly(T) tract (acting as a termination signal for RNA pol Ill), and the G/C content is advised to be between 35-55%. shRNAs should comprise sense and antisense sequences (advised to each be 19-21 nt in length) separated by loop structure, and a 3′ AAAA overhang. Effective loop structures are suggested to be 3-9 nt in length. It is suggested to follow the sense-loop-antisense order in designing the shRNA cassette and to avoid 5′ overhangs in the shRNA construct. Finally, several companies commercially offer premade siRNAs and shRNAs.

### GAPMERs

In a particular embodiments, the antisense oligonucleotide of the invention or the contiguous nucleotide sequence thereof is a gapmer. A gapmer or gapmer oligonucleotide comprises at least three distinct structural regions: a 5′-flank, a gap and a 3′-flank or F-G-F′ in the ′5->3′ orientation. The “gap” region (G) comprises a stretch of contiguous DNA nucleotides which enable the oligonucleotide to recruit RNase H. The gap region is flanked by a 5′ flanking region (F) comprising one or more sugar modified nucleosides, and by a 3′ flanking region (F′) comprising one or more sugar modified nucleosides. The one or more sugar modified nucleosides in region F and F′ enhance the affinity of the oligonucleotide for the target nucleic acid (i.e. are affinity enhancing sugar modified nucleosides). In some embodiments, the one or more sugar modified nucleosides in region F and F′ are 2′ sugar modified nucleosides, such as independently selected from LNA and 2′-MOE.

In a gapmer design, the 5′ and 3′ most nucleosides of the gap region are DNA nucleosides, and are positioned adjacent to a sugar modified nucleoside of the 5′ (F) or 3′ (F′) region respectively. The flanks may further be defined by having at least one sugar modified nucleoside at the end most distant from the gap region, i.e. at the 5′ end of the 5′ flank and at the 3′ end of the 3′ flank. Regions F-G-F′ form a contiguous nucleotide sequence. Antisense oligonucleotides of the invention, or the contiguous nucleotide sequence thereof, may comprise a gapmer region of formula F-G-F′.

Regions F and F′ independently comprise 1-8 contiguous nucleosides, of which 1-5 independently can be 2′ sugar modified and defines the 5′ and 3′ end of the F and F′ region. Region F is positioned immediately adjacent to the 5′ DNA nucleoside of region G. In one embodiment, the 3′ most nucleoside of region F is a sugar modified nucleoside, for example a 2′ substituted nucleoside, such as a MOE nucleoside, or an LNA nucleoside. Region F′ is positioned immediately adjacent to the 3′ DNA nucleoside of region G. In one embodiment, the 5′ most nucleoside of region F′ is a sugar modified nucleoside, for example a 2′ substituted nucleoside, such as a MOE nucleoside, or an LNA nucleoside.

In one embodiment, region F is between 1 and 8 contiguous nucleotides in length, such as 2-6, such as 3-4 contiguous nucleotides in length. In particular embodiments, the 5′ most nucleoside of region F is a sugar modified nucleoside. In some embodiments the two 5′ most nucleoside of region F are sugar modified nucleoside. In some embodiments the 5′ most nucleoside of region F is an LNA nucleoside. In some embodiments the two 5′ most nucleoside of region F are LNA nucleosides. In some embodiments the two 5′ most nucleoside of region F are LNA nucleosides. In some embodiments the two 5′ most nucleoside of region F are 2′ substituted nucleoside nucleosides, such as two 3′ MOE nucleosides. In some embodiments the 5′ most nucleoside of region F is a 2′ substituted nucleoside, such as a MOE nucleoside.

In one embodiment, region F′ is between 2 and 8 contiguous nucleotides in length, such as 3-6, such as 4-5 contiguous nucleotides in length. Particularly embodiments provide that the 3′ most nucleoside of region F′ is a sugar modified nucleoside. In some embodiments the two 3′ most nucleoside of region F′ are sugar modified nucleoside. In some embodiments the two 3′ most nucleoside of region F′ are LNA nucleosides. In some embodiments the 3′ most nucleoside of region F′ is an LNA nucleoside. In some embodiments the two 3′ most nucleoside of region F′ are 2′ substituted nucleoside nucleosides, such as two 3′ MOE nucleosides. In some embodiments the 3′ most nucleoside of region F′ is a 2′ substituted nucleoside, such as a MOE nucleoside.

It should be noted that when the length of region F or F′ is one, it is preferably an LNA nucleoside.

In some embodiments, region F and F′ independently consists of or comprises a contiguous sequence of sugar modified nucleosides. In some embodiments, the sugar modified nucleosides of region F may be independently selected from 2′-O-alkyl-RNA units, 2′-O-methyl-RNA, 2′-amino-DNA units, 2′-fluoro-DNA units, 2′-alkoxy-RNA, MOE units, LNA units, arabino nucleic acid (ANA) units and 2′-fluoro-ANA units. In some embodiments, region F and F′ independently comprises both LNA and a 2′ substituted modified nucleosides. In some embodiments, region F and F′ consists of only one type of sugar modified nucleosides, such as only MOE or only beta-D-oxy LNA or only ScET. Such designs are also termed uniform flanks or uniform gapmer design.

In some embodiments, all the nucleosides of region F or F′, or F and F′ are LNA nucleosides, such as independently selected from beta-D-oxy LNA, ENA or ScET nucleosides. In some embodiments region F consists of 1-5, such as 2-4, such as 3-4 such as 1, 2, 3, 4 or 5 contiguous LNA nucleosides. In some embodiments, all the nucleosides of region F and F′ are LNA nucleosides or beta-D-oxy LNA nucleosides. In some embodiments, all the nucleosides of region F or F′, or F and F′ are 2′ substituted nucleosides, such as OMe or MOE nucleosides. In some embodiments region F consists of 1, 2, 3, 4, 5, 6, 7, or 8 contiguous OMe or MOE nucleosides. In some embodiments only one of the flanking regions can consist of 2′ substituted nucleosides, such as OMe or MOE nucleosides. In some embodiments it is the 5′ (F) flanking region that consists 2′ substituted nucleosides, such as OMe or MOE nucleosides whereas the 3′ (F′) flanking region comprises at least one LNA nucleoside, such as beta-D-oxy LNA nucleosides or cET nucleosides. In some embodiments it is the 3′ (F′) flanking region that consists 2′ substituted nucleosides, such as OMe or MOE nucleosides whereas the 5′ (F) flanking region comprises at least one LNA nucleoside, such as beta-D-oxy LNA nucleosides or cET nucleosides.

In some embodiments the 5′ most and the 3′ most nucleosides of region F and F′ are LNA nucleosides, such as beta-D-oxy LNA nucleosides or ScET nucleosides.

Region G comprises or consists of between 5 and 18 nucleosides which are capable of recruiting RNase H. Suitably gapmers may have a gap region of at least 5 or 6 contiguous DNA nucleosides, such as 5-16 contiguous DNA nucleosides, such as 6-15 contiguous DNA nucleosides, such as 7-14 contiguous DNA nucleosides, such as 8-12 contiguous DNA nucleotides, such as 8-12 contiguous DNA nucleotides in length. The gap region G may, in some embodiments consist of 6, 7, 8, 9, 10, 11, 12, 13, 14, 15 or 16 contiguous DNA nucleosides. One or more cytosine (C) DNA in the gap region may in some instances be methylated (e.g. when a DNA c is followed by a DNA g) such residues are either annotated as 5-methyl-cytosine (meC). In some embodiments the gap region G may consist of 6, 7, 8, 9, 10, 11, 12, 13, 14, 15 or 16 contiguous phosphorothioate linked DNA nucleosides. In some embodiments, all internucleoside linkages in the gap are phosphorothioate linkages

The overall length of the gapmer design F-G-F′ may be, for example 12 to 32 nucleosides, such as 13 to 24, such as 14 to 22 nucleosides, such as from 14 to 17, such as 16 to 18 nucleosides.

In some embodiments, the internucleoside linkage between region F and region G is a phosphorothioate internucleoside linkage. In some embodiments, the internucleoside linkage between region F′ and region G is a phosphorothioate internucleoside linkage. In some embodiments, the internucleoside linkages between the nucleosides of region F or F′, F and F′ are phosphorothioate internucleoside linkages.

In some embodiments, the gapmer of the invention is a LNA Gapmer. An LNA gapmer is a gapmer wherein either one or both of region F and F′ comprises or consists of LNA nucleosides. A beta-D-oxy gapmer is a gapmer wherein either one or both of region F and F′ comprises or consists of beta-D-oxy LNA nucleosides.

In other embodiments, the gapmer of the invention is a MOE Gapmer. A MOE gapmers is a gapmer wherein regions F and F′ consist of MOE nucleosides. In some embodiments the MOE gapmer is of design [MOE]1-8-[Region G]-[MOE] 1-8, such as [MOE]2-7-[Region G]5-16-[MOE] 2-7, such as [MOE]3-6-[Region G]-[MOE] 3-6, wherein region G is as defined in the Gapmer definition. MOE gapmers with a 5-10-5 design (MOE-DNA-MOE) have been widely used in the art.

In particular embodiments, the gapmer of the invention can also comprise a region D′ and/or D″. Said regions refer to additional 5′ and/or 3′ nucleosides which may or may not be fully complementary to the target nucleic acid. The addition of region D′ or D″ may be used for the purpose of joining the contiguous nucleotide sequence, such as the gapmer, to a conjugate moiety or another functional group. When used for joining the contiguous nucleotide sequence with a conjugate moiety is can serve as a biocleavable linker. Alternatively, it may be used to provide exonuclease protection or for ease of synthesis or manufacture. Region D′ and D″ can be attached to the 5′ end of region F or the 3′ end of region F′, respectively to generate designs of the following formulas D′-F-G-F′, F-G-F′-D″ or D′-F-G-F′-D″. In this instance the F-G-F′ is the gapmer portion of the oligonucleotide and region D′ or D″ constitute a separate part of the oligonucleotide.

Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these. The D′ or D′ region may serve as a nuclease susceptible biocleavable linker (see definition of linkers). In some embodiments the additional 5′ and/or 3′ end nucleotides are linked with phosphodiester linkages, and are DNA or RNA.

CRISPR gRNA

Another recent genome editing technology is the CRISPR/Cas system, which can be used to achieve RNA-guided genome engineering. CRISPR interference is a genetic technique which allows for sequence-specific control of gene expression in prokaryotic and eukaryotic cells. It is based on the bacterial immune system-derived CRISPR (clustered regularly interspaced palindromic repeats) pathway. Recently, it was demonstrated that the CRISPR-Cas editing system can also be used to target RNA. It has been shown that the Class 2 type VI-A CRISPR-Cas effector C2c2 can be programmed to cleave single stranded RNA targets carrying complementary protospacers (Abudayyeh et al 2016 Science 353/science.aaf5573). C2c2 is a single-effector endoRNase mediating ssRNA cleavage once it has been guided by a single crRNA guide toward the target RNA. Hence, the invention disclosed herein can also be applied to develop gRNAs specifically reducing the expression of SYNGR-3 using the CRISPR/Cas system. Therefore, the application also provides that any of the oligonucleotides herein provided is a gRNA or CRISPR gRNA, more particularly a gRNA or CRISPR gRNA is provided with at least 90% complementary to a region of Synaptogyrin-3 as depicted in SEQ ID No. 1 or 3, wherein the region is selected from SEQ ID No. 38-54, SEQ ID No. 69-79, SEQ ID No. 100-104, SEQ ID No. 108-109 or SEQ ID No. 115-121. In a particular embodiment, said gRNA is 10 to 50 or 10 to 40 or 10 to 30 nucleotides in length. In another particular embodiment, said gRNA comprises a contiguous nucleotide sequence of at least 10 contiguous nucleotides in length which are at least 90% complementary to a region of Synaptogyrin-3 as depicted in SEQ ID No. 1 or 3, wherein the region is selected from SEQ ID No. 38-54, SEQ ID No. 69-79, SEQ ID No. 100-104, SEQ ID No. 108-109 or SEQ ID No. 115-121.

In some aspects, the gRNA comprises a contiguous nucleotide sequence of at least 10 contiguous nucleotides in length comprising a sequence selected from the group consisting of SEQ ID No. 7, 16-18, 24 and 55-68. In some aspects, the gRNA of the present disclosure comprises a sequence that overlaps with 9, 10, 11, 12, 13, 14, 15, or 16 nucleobase subsequence from a sequence selected from the group consisting of SEQ ID NO: 7, 16-18, 24, 55-68.

### Pharmaceutical Salt

The nucleic acid molecules or oligonucleotides according to the present invention may exist in the form of their pharmaceutically acceptable salts. The term “pharmaceutically acceptable salt” refers to conventional acid-addition salts or base-addition salts that retain the biological effectiveness and properties of the nucleic acid molecules or oligonucleotides of the present invention and are formed from suitable non-toxic organic or inorganic acids or organic or inorganic bases. Acid-addition salts include for example those derived from inorganic acids such as hydrochloric acid, hydrobromic acid, hydroiodic acid, sulfuric acid, sulfamic acid, phosphoric acid and nitric acid, and those derived from organic acids such as p-toluene sulfonic acid, salicylic acid, methane sulfonic acid, oxalic acid, succinic acid, citric acid, malic acid, lactic acid, fumaric acid, and the like. Base-addition salts include those derived from ammonium, potassium, sodium and, quaternary ammonium hydroxides, such as for example, tetramethyl ammonium hydroxide. The chemical modification of a pharmaceutical compound into a salt is a technique well known to pharmaceutical chemists in order to obtain improved physical and chemical stability, hygroscopicity, flowability and solubility of compounds. It is for example described by Bastin (2000 Organic Process Research & Development 4:427-435) or in Ansel (1995 In: Pharmaceutical Dosage Forms and Drug Delivery Systems, 6th ed., pp. 196 and 1456-1457). For example, the pharmaceutically acceptable salt of the nucleic acid molecules or oligonucleotides provided herein may be a sodium salt. Provided herein is a pharmaceutically acceptable salt of the nucleic acid molecules or oligonucleotides described herein. In one embodiment, the pharmaceutically acceptable salt is a sodium or a potassium salt.

### Pharmaceutical Composition

In another aspect, the invention provides pharmaceutical compositions comprising any of the nucleic acid molecules or oligonucleotides described herein or salts thereof and a pharmaceutically acceptable diluent, carrier, salt and/or adjuvant. A pharmaceutically acceptable diluent includes phosphate-buffered saline (PBS) and pharmaceutically acceptable salts include, but are not limited to sodium and potassium salts. In some embodiments the pharmaceutically acceptable diluent is sterile phosphate buffered saline. In some embodiments the nucleic acid molecules or oligonucleotides of the application are used in the pharmaceutically acceptable diluent at a concentration of 50-300 mM solution.

Suitable formulations for use in the present invention are found in Remington's Pharmaceutical Sciences, Mack Publishing Company, Philadelphia, Pa., 17th ed., 1985. For a brief review of methods for drug delivery, see e.g. Langer (1990 Science 249:1527-1533). Non-limiting examples of pharmaceutically acceptable diluents, carriers, adjuvants, suitable dosages, formulations, administration routes, compositions, dosage forms, combinations with other therapeutic agents, pro-drug formulations are provided in WO2007/031091. The nucleic acid molecules or oligonucleotides of the application or salts thereof may be mixed with pharmaceutically acceptable active or inert substances for the preparation of pharmaceutical compositions or formulations. Compositions and methods for the formulation of pharmaceutical compositions are dependent upon a number of criteria, including but not limited to route of administration, extent of disease, or dose to be administered. Pharmaceutical compositions comprising any of the nucleic acid molecules or oligonucleotides of the application or salts thereof may be sterilized by conventional sterilization techniques or may be sterile filtered. The resulting aqueous solutions may be packaged for use as is or lyophilized, the lyophilized preparation being combined with a sterile aqueous carrier prior to administration.

The pH of the preparations typically will be between 3 and 11, more particularly between 5 and 9 or between 6 and 8, most particularly between 7 and 8, such as 7 to 7.5. The resulting compositions in solid form may be packaged in multiple single dose units, each containing a fixed amount of the nucleic acid molecules or oligonucleotides of the application or salts thereof, such as in a sealed package of tablets or capsules. The composition in solid form can also be packaged in a container for a flexible quantity.

### Tauopathic Disorders

It was previously shown that SYNGR-3 interacts with pathological Tau at the presynapse and that reducing the level of SYNGR-3 rescued Tau-induced defects in vesicle mobility and neurotransmitter release (McInnes et al 2018 Neuron 97:823-835). Inhibiting the expression of synaptogyrin-3 to reduce binding between synaptogyrin-3 and (the N-terminal sequence of) the tau protein is thus at the heart of the current invention. Therefore in a second aspect, any of the nucleic acid molecules or oligonucleotides described in current application is provided for use as a medicament. More particularly for use to treat tauopathies.

Tauopathies are a diverse group of disorders all having in common their association with prominent accumulation of intracellular tau protein. The tau protein is abundantly expressed in the central nervous system. The group of tauopathies is growing as recently Huntington disease (Fernandez-Nogales et al 2014 Nat Med 20:881-885) and chronic traumatic encephalopathy (CTE; McKee et al 2009 J Neuropathol Exp Neurol 68, 709-735) were added.

Different classifications of tauopathies exist. In one classification system, tauopathic disorders are divided in predominant Tau pathologies, tauopathies associated with amyloid deposition and tauopathies associated with another pathology (Williams et al 2006 Intern Med J 36:652-660). Predominant Tau pathologies include progressive supranuclear palsy (PSP), progressive supranuclear palsy-parkinsonism (PSP-P), Richardson's syndrome, argyrophilic grain disease, corticobasal degeneration, Pick's disease, frontotemporal dementia with parkinsonism associated with chromosome 17 (FTDP-17), post-encephalitic parkinsonism, Parkinson's disease complex of Guam, and Guadeloupean parkinsonism. Tauopathic disorders associated with amyloid deposition include Alzheimer's disease, Down's syndrome, dementia pugilistica, familial British dementia and familial Danish dementia. Tauopathic disorders associated with another pathology include myotonic dystrophy, Hallevorden-Spatz disease, and Niemann Pick type C.

Another classification is based on the isoform type found in the aggregates although overlaps may exist: 4R tauopathies include progressive supranuclear palsy (PSP), corticobasal degeneration, tangle predominant dementia, and argyrophilic grain disease. 3R tauopathies include Pick disease, and 3R+4R tauopathies include Alzheimer's disease (Dickson et al 2011 J Mol Neurosci 45:384-389; Murray et al 2014 Alzheimer's Res Ther 6:1). The tau protein is discussed herein in more detail further below.

Further tauopathies include tangle-only dementia, white matter tauopathy with globular glial inclusions, subacute sclerosing panencephalitis, SLC9A6-related mental retardation, non-Guamanian motor neuron disease with neurofibrillary tangles, neurodegeneration with brain iron accumulation, Gerstmann-Straussler-Scheinker disease, frontotemporal lobar degeneration, diffuse neurofibrillary tangles with calcification, chronic traumatic encephalopathy, amyotrophic lateral sclerosis of Guam, amyotrophic lateral sclerosis and parkinsonism-dementia complex, prion protein cerebral amyloid angiopathy, and progressive subcortical gliosis (Murray et al 2014 Alzheimer's Res Ther 6:1; Spillantini & Goedert 2013 Lancet Neurol 12:609-622).

Symptoms of tauopathic disorders include clinical or pathological symptoms such as mild cognitive impairment, dementia, cognitive decline (e.g. apathy, impairment in abstract thought), decline of motor function (causing e.g. postural instability, tremor or dystonia), oculomotor and bulbar dysfunction. Criteria for diagnosing dementia are outlined in e.g. the Diagnostic and Statistical Manual of Mental Disorders (DSM) or in the International Classification of Disease (ICD) and are subject to regular updates. The type of clinical symptoms depends on which region of the brain is affected by the tauopathy and explains why Alzheimer's disease is mainly a dementing disease and why Parkinson's disease is mainly affecting movement. Stereotypical temporospatial propagation of tau inclusions creates a consistent pattern of brain lesions in at least Alzheimer's disease and argyrophilic grain disease. The spreading may in part occur in a trans-synaptic manner (Spillantini & Goedert 2013 Lancet Neurol 12:609-622; Liu et al 2012 PloS One 7:e31802). Molecular symptoms of tauopathic disorders include synaptic dysfunction (in particular pre-synaptic dysfunction), neurotoxicity, neuronal degeneration, neuronal dysfunction, synapse loss and amyloid deposition.

Given that the nucleic acid molecules or oligonucleotides herein described are able to target synaptogyrin-3 and reduce its expression, any of said nucleic acid molecules or oligonucleotides is thus applicable for use as a medicament. In one embodiment thereto, any of the nucleic acid molecules or oligonucleotides herein described is provided for use in (a method for) treating or inhibiting progression of a tauopathic disorder or for use in (a method for) treating or inhibiting a symptom of a tauopathic disorder. In particular, the nucleic acid molecules or oligonucleotides of the invention are inhibitors of human synaptogyrin-3 expression. The expression or function of synaptogyrin-3 is (partially) inhibited such as to restore pathological Tau-induced presynaptic dysfunction. In the methods for treating or inhibiting progression of a tauopathic disorder or a symptom of a tauopathic disorder, any of the nucleic acid molecules or oligonucleotides herein described is administered to a subject in need thereof (a subject suffering of or displaying a tauopathy or symptom thereof) in an effective amount, i.e. in an amount sufficient to treat or to inhibit progression of a tauopathic disorder or a symptom of a tauopathic disorder.

For the purpose of treating, preventing or inhibiting (progression of) an intended disease or disorder, and in method for treating, preventing or inhibiting (progression of) an intended disease or disorder, an effective amount of the therapeutic compound is administered to a subject in need thereof. An “effective amount” of an active substance in a composition is the amount of said substance required and sufficient to elicit an adequate response in treating, preventing, inhibiting (progression of) the intended or targeted medical indication. It will be clear to the skilled artisan that such response may require successive (in time) administrations with the composition as part of an administration scheme. The effective amount may vary depending on the nature of the compound, the route of administration of the compound (crossing of the blood-brain barrier and the cell membrane are potential barriers to be taken by oligonucleotides as described herein), the health and physical condition of the individual to be treated, the age of the individual to be treated (e.g. dosing for infants may be lower than for adults) the taxonomic group of the individual to be treated (e.g. human, non-human primate, primate, etc.), the capacity of the individual's system to respond effectively, the degree of the desired response, the formulation of the active substance, the treating doctor's assessment and other relevant factors. The effective amount further may vary depending on whether it is used in monotherapy or in combination therapy. Determination of an effective amount of a compound usually follows from pre-clinical testing in a representative animal or in vitro model (if available) and/or from dose-finding studies in early clinical trials.

Any of the nucleic acid molecules or oligonucleotides described herein is provided for use in (a method for) treating or inhibition progression of a tauopathic disorder wherein the tauopathic disorder is selected from the group consisting of Alzheimer's disease, progressive supranuclear palsy (PSP), progressive supranuclear palsy-parkinsonism (PSP-P), Richardson's syndrome, argyrophilic grain disease, corticobasal degeneration Pick's disease, frontotemporal dementia with parkinsonism associated with chromosome 17 (FTDP-17), post-encephalitic parkinsonism, Parkinson's disease complex of Guam, Guadeloupean parkinsonism, Huntington disease, Down's syndrome, dementia pugilistica, familial British dementia, familial Danish dementia, myotonic dystrophy, Hallevorden-Spatz disease, Niemann Pick type C, chronic traumatic encephalopathy, tangle-only dementia, white matter tauopathy with globular glial inclusions, subacute sclerosing panencephalitis, SLC9A6-related mental retardation, non-Guamanian motor neuron disease with neurofibrillary tangles, neurodegeneration with brain iron accumulation, Gerstmann-Straussler-Scheinker disease, frontotemporal lobar degeneration, diffuse neurofibrillary tangles with calcification, chronic traumatic encephalopathy, amyotrophic lateral sclerosis of Guam, amyotrophic lateral sclerosis and parkinsonism-dementia complex, prion protein cerebral amyloid angiopathy, and progressive subcortical gliosis.

Any of the nucleic acid molecules or oligonucleotides described herein is thus likewise applicable for use in (a method for) treating or inhibition progression of a symptom of tauopathic disorder selected from the group of mild cognitive impairment, dementia, cognitive decline, decline of motor function, oculomotor and bulbar dysfunction, synaptic dysfunction, neurotoxicity, neuronal degeneration, neuronal dysfunction, synapse loss, and amyloid deposition. In particular, in relation to synaptic dysfunction it concerns pre-synaptic dysfunction.

“Treatment” refers to any rate of reduction or retardation of the progress of the disease or disorder compared to the progress or expected progress of the disease or disorder when left untreated. More desirable, the treatment results in no/zero progress of the disease or disorder (i.e. “inhibition” or “inhibition of progression”) or even in any rate of regression of the already developed disease or disorder. Tauopathies are in general progressive disorders, and progression may imply propagation of pathological tau protein (Asai et al 2015 Nat Neurosci 18:1584-1593; deCalignon et al 2012 Neuron 73:685-697).

“Reduction” or “reducing” as used herein refers to a statistically significant reduction. More particularly, a statistically significant reduction upon administering the oligonucleotide of the invention compared to a control situation wherein the oligonucleotide is not administered. In a particular embodiment, said statistically significant reduction is an at least 25%, 30%, 35%, 40%, 45% or 50% reduction compared to the control situation.

The application also provides methods of treating or inhibiting progression of a symptom of a tauopathic disorder, the method comprises the step of administering any of the nucleic acid molecules or oligonucleotides herein described to a subject in need thereof.

In one embodiment, a method of reducing the expression level of synaptogyrin-3 in a subject is provided, comprising the step of administering any of the nucleic acid molecules or oligonucleotides herein described to the subject.

### Diagnosis of Tauopathic Disorders

Magnetic resonance imaging (MRI) in itself allows for radiologic determination of brain atrophy. Midbrain atrophic signs such as the Hummingbird or Penguin silhouette are for instance indicators of progressive supranuclear palsy (PSP). Determination of tau protein content in the cerebrospinal fluid (CSF) may also serve as an indicator of tauopathies. The ratio between the 33 kDa/55 kDa tau-forms in CSF was e.g. found to be reduced in a patients with PSP (Borroni et al 2008 Neurology 71:1796-1803). Recently, in vivo imaging techniques of neurodegeneration have become available. Such techniques can clearly support the clinical diagnosis of neurodegenerative diseases in general and of tauopathies in particular. In vivo diagnosis of tauopathies benefits from the existence of Tau imaging ligands detectable by positron emission tomography (PET), and include the radiotracers 2-(1-(6-((2-[¹⁸F]fluoroethyl) (methyl) amino)-2-naphthyl)ethylidene) malononitrile ([¹⁸F]FDDNP), 2-(4-aminophenyl)-6-(2-([¹⁸F]fluoroethoxy))quinolone ([¹⁸F]THK523), and [¹⁸F]T807 and [¹⁸F]T808 (Murray et al 2014 Alzheimer's Res Ther 6:1). In addition, MRI can be used to detect tauopathies, and PET imaging with fluorodeoxyglucose (FDG, ¹⁸F agent) is indicative of synaptic activity (Murray et al 2014 Alzheimer's Res Ther 6:1). Beta-amyloid, that can be detected in vivo, e.g. by using florbetapir (or other amyloid markers) in combination with PET, proved to be an accurate biomarker for at least Alzheimer's disease (Clark et al 2011 J Am Med Assoc 305:275-283) and the florbetapir-PET technique received FDA approval in 2012. The availability of in vivo tauopathy detection techniques is further supportive for selecting subjects that can benefit from synaptogyrin-3 inhibitory therapies as described herein.

### Inhibition of Synaptogyrin-3

By using the nucleic acid molecules or oligonucleotides of the invention, inhibition of synaptogyrin-3 is obtained at the expression level. As demonstrated previously (see WO2019/016123), partial inhibition of synaptogyrin-3 is sufficient to restore pathological Tau-induced presynaptic dysfunction. As such, inhibition of synaptogyrin-3 implies several possible levels of inhibition, e.g. at least 20%, at least 30%, at least 40%, at least 50%, at least 60%, at least 70%, at least 80%, at least 90%, at least 95% or even 100% inhibition.

### Administrating the Nucleic Acid Molecules of the Invention

The nucleic acid molecules or the oligonucleotides of the present invention may be administered via intravenous, subcutaneous, intramuscular, intracerebral, intracerebroventricular, intraventricular, intraocular, or intrathecal administration. In some embodiments, the administration is via intrathecal administration. “Administering” as used herein, means to give a composition comprising a composition disclosed herein to a subject via a pharmaceutically acceptable route.

SYNGR-3 gene inactivation, i.e. inhibition of expression of the target gene, can be also achieved through the creation of transgenic organisms expressing one of the oligonucleotides of the invention (e.g. siRNA), or by administering said inhibitor to the subject (see Examples). The nature of the inhibitor (siRNA, shRNA, gapmer, etc) and whether the effect is achieved by incorporating the oligonucleotide into the subject's genome or by administering the oligonucleotide is not vital to the invention, as long as said oligonucleotide reduces the level of Syngr-3 transcripts. An oligonucleotide construct can be delivered, for example as an expression plasmid, which when transcribed in the cell, produces the oligonucleotide that is complementary to at least a unique portion of the cellular SYNGR-3 RNA. Alternatively, oligonucleotide inhibitors such as siRNA can also be expressed from recombinant circular or linear DNA plasmids using any suitable promoter. Suitable promoters for expressing these inhibitors targeted against SYNGR-3 from a plasmid include, for example the U6 or H1 RNA polymerase 11 promoter sequences and the cytomegalovirus promoter. Selection of other suitable promoters is within the skill in the art. Non-limiting examples are neuronal-specific promoters, glial cell specific promoters, the human synapsin 1 gene promoter, the Hb9 promotor or the promoters disclosed in U.S. Pat. No. 7,341,847B2.

The recombinant plasmids comprising any of the nucleic acid molecules or oligonucleotides of the invention can also comprise inducible or regulatable promoters for expression of the nucleic acid molecule or oligonucleotide in a particular tissue or in a particular intracellular environment. The nucleic acid molecule or oligonucleotide expressed from recombinant plasmids can either be isolated from cultured cell expression systems by standard techniques, or can be expressed intracellularly, e.g. in brain tissue or in neurons. Nucleic acid molecules or oligonucleotides can also be expressed intracellularly from recombinant viral vectors. The recombinant viral vectors comprise sequences encoding the nucleic acid molecules or oligonucleotides of the invention and any suitable promoter for expressing them. The nucleic acid molecules or oligonucleotides will be administered in an “effective amount” which is an amount sufficient to cause a statistically significant reduction of the Syngr-3 transcript. Generally, an effective amount of a nucleic acid molecule or oligonucleotide targeting Syngr-3 transcripts comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of inhibitor can be administered.

shRNAs for example can be introduced into the nuclei of target cells using a vector (e.g. bacterial or viral) that optionally can stably integrate into the genome. shRNAs are usually transcribed from vectors, e.g. driven by the Pol Ill U6 promoter or H1promoter. Vectors allow for inducible shRNA expression, e.g. relying on the Tet-on and Tet-off inducible systems commercially available, or on a modified U6 promoter that is induced by the insect hormone ecdysone. A Cre-Lox recombination system has been used to achieve controlled expression in mice. Synthetic shRNAs can be chemically modified to affect their activity and stability. Plasmid DNA or dsRNA can be delivered to a cell by means of transfection (lipid transfection, cationic polymer-based nanoparticles, lipid or cell-penetrating peptide conjugation) or electroporation. Viral vectors include lentiviral, retroviral, adenoviral and adeno-associated viral vectors.

### Drug Administration Across Blood-Brain Barrier

In some aspects, the oligonucleotides of the present disclosure are administered across the blood-brain barrier. The blood-brain barrier (BBB) is a protective layer of tightly joined cells that lines the blood vessels of the brain which prevents entry of harmful substances (e.g. toxins, infectious agents) and restricts entry of (non-lipid) soluble molecules that are not recognized by specific transport carriers into the brain. This poses a challenge in the delivery of drugs, such as the synaptogyrin-3 inhibitors described herein, to the central nervous system/brain in that drugs transported by the blood not necessarily will pass the blood-brain barrier. Although the BBB often is to some degree affected or broken down in case of a tauopathic disorder, it may be needed to rely on a means to enhance permeation of the BBB for a candidate drug for treating a tauopathic disorder to be able to enter the affected brain cells. Thus, in some aspects, the oligonucleotides of the present disclose are formulated, conjugated, or carried by vectors, polymers, cells, or devices, to name a few alternatives, that allow the oligonucleotides to cross the BBB. Several options are nowadays available for delivery of drugs across the BBB (Peschillo et al 2016 J Neurointervent Surg 8:1078-1082; Miller & O'Callaghan 2017 Metabolism 69:S3-S7; Drapeau & Fortin 2015 Current Cancer Drug Targets 15:752-768).

Drugs can be directly injected into the brain (invasive strategy) or can be directed into the brain after BBB disruption with a pharmacological agent (pharmacologic strategy). In some aspects, an oligonucleotide of the present disclosure can be directly injected into the brain, e.g., using a needle or a catheter. In some aspects, an oligonucleotide of the present disclosure can be directed into the brain by BBB disruption with a pharmacological agent. Invasive means of BBB disruption are associated with the risk of hemorrhage, infection or damage to diseased and normal brain tissue from the needle or catheter. Direct drug deposition may be improved by the technique of convection-enhanced delivery. Accordingly, in some aspects, an oligonucleotide of the present disclosure can be administered via convection-enhanced delivery.

Longer term delivery of a therapeutic protein (e.g. a neurotrophic factor or nerve growth factor, or a proteinaceous synaptogyrin-3 inhibitor as describe herein) can be achieved by implantation of genetically modified stem cells, by recombinant viral vectors, by means of osmotic pumps, or by means of incorporating the therapeutic drug in a polymer (slow release; can be implanted locally). Thus, in some aspects, an oligonucleotide of the present disclosure can be administered, e.g., by implantation of genetically modified cells (e.g., stem cells), recombinant vectors (e.g., viral vectors), delivery devices (e.g., pumps such as osmotic pumps), or incorporation in a polymer.

Pharmacologic BBB disruption has the drawback of being non-selective and can be associated with unwanted effects on blood pressure and the body's fluid balance. This is circumvented by targeted or selective administration of the pharmacologic BBB disrupting agent. As an example, intra-arterial cerebral infusion of an antibody (bevacizumab) in a brain tumor was demonstrated after osmotic disruption of the BBB with mannitol (Boockvar et al. 2011, J Neurosurg 114:624-632); other agents capable of disrupting the BBB pharmacologically include bradykinin and leukotriene C4 (e.g. via intracarotid infusion; Nakano et al. 1996, Cancer Res 56:4027-4031). Thus, in some aspects, the oligonucleotides of the present disclosure are formulated in combination with a pharmacologic BBB disrupting agent. In some aspects, the oligonucleotides of the present disclosure are administered in combination with a pharmacologic BBB disrupting agent. In some aspects, the pharmacologic BBB disrupting agent is administered prior to the administration of the oligonucleotide of the present disclosure. In some aspects, the pharmacologic BBB disrupting agent is administered concurrently to the administration of the oligonucleotide of the present disclosure. In some aspects, the pharmacologic BBB disrupting agent is administered subsequently to the administration of the oligonucleotide of the present disclosure. In some aspects, the pharmacologic BBB disrupting agent comprises mannitol, bradykinin, leukotriene C4, or a combination thereof.

BBB transcytosis and efflux inhibition are other strategies to increase brain uptake of drugs supplied via the blood. Using transferrin or transferrin-receptor antibodies as carrier of a drug is one example of exploiting a natural BBB transcytosis process (Friden et al. 1996, J Pharmacol Exp Ther 278:1491-1498). Exploiting BBB transcytosis for drug delivery is also known as the molecular Trojan horse strategy. In some aspects, the oligonucleotides of the present disclosure are conjugated to carrier, e.g., transferrin or a transferrin-receptor antibody. In some aspects, the oligonucleotides of the present disclosure are conjugated or formulated to transverse the BBB via transcytosis. Another mechanism underlying BBB, efflux pumps or ATP-binding cassette (ABC) transporters (such as breast cancer resistance protein (BCRP/ABCG2) and P-glycoprotein (Pgp/MDR1/ABCB1)), can be blocked in order to increase uptake of compounds (e.g. Carcaboso et al. 2010, Cancer Res 70:4499-4508). In some aspects, the oligonucleotides of the present disclosure can be formulated in combination with a compound that can block an ABC transporter, a compound that can block P-glycoprotein, or a combination thereof.

Kumar et al (2007 Nature 448:39-43) demonstrated uptake of siRNAs in the brain after coupling to a 29-amino acid peptide derived from rabies virus glycoprotein (RVG) which is specifically binding the acetylcholine receptor. In some aspects, the oligonucleotides of the present disclosure are conjugated to RVG.

Therapeutic drugs can alternatively be loaded in liposomes to enhance their crossing of the BBB, an approach also known as liposomal Trojan horse strategy. Thus, in some aspects, the oligonucleotides of the present disclosure are formulated in liposomes, e.g., liposomes for use in a liposomal Trojan horse strategy.

Especially in the field of treating cognitive and neurodegenerative disorders there has been quite some interest in intranasal delivery of drugs (e.g. Muhs et al. 2007, Proc Natl Acad Sci USA 104:9810-9815; Kao et al. 2000, Pharm Res 17:978-984; Hanson & Frey 2008, BMC Neurosci 9 (Suppl3): 55). This strategy is based on the trigeminal and olfactory nerves that innervate the nasal epithelium, representing direct connections between the external environment and the brain. Thus, in some aspects, the oligonucleotides of the present disclosure are formulated for intranasal delivery.

A more recent and promising avenue for delivering therapeutic drugs to the brain consists of (transient) BBB disruption by means of ultrasound, more particularly focused ultrasound (FUS; Miller et al. 2017, Metabolism 69:S3-S7). Besides being non-invasive, this technique has, often in combination with realtime imaging, the advantage of precise targeting to a diseased area of the brain. Therapeutic drugs can be delivered in e.g. microbubbles e.g. stabilized by an albumin or other protein, a lipid, or a polymer. Therapeutic drugs can alternatively, or in conjunction with microbubbles, be delivered by any other method, and subsequently FUS can enhance local uptake of any compound present in the blood (e.g. Nance et al. 2014, J Control Release 189:123-132). Just one example is that of FUS-assisted delivery of antibodies directed against toxic amyloid-beta peptide with demonstration of reduced pathology in mice (Jordao et al. 2010, PloS One 5:e10549). Microbubbles with a therapeutic drug load can also be induced to burst (hyperthermic effect) in the vicinity of the target cells by means of FUS, and when driven by e.g. a heat shock protein gene promoter, localized temporary expression of a therapeutic protein can be induced by ultrasound hyperthermia (e.g. Lee Titsworth et al. 2014, Anticancer Res 34:565-574). Alternatives for ultrasound to induce the hyperthermia effect are microwaves, laser-induced interstitial thermotherapy, and magnetic nanoparticles (e.g. Lee Titsworth et al. 2014, Anticancer Res 34:565-574). Thus, in some aspects, the oligonucleotides of the present disclosure are formulated for FUS-mediated delivery.

### Intracellular Drug Administration

In some aspects, the oligonucleotides of the present disclosure are formulated for intracellular administration. Besides the need to cross the BBB, drugs targeting disorders of the central nervous system, such as the synaptogyrin-3 inhibitors described herein, may also need to cross the cellular barrier. Although most antisense oligonucleotides are readily taken up by neurons and glia after reaching the nervous system, it can be advantageous to use facilitators of intracellular drug uptake.

One solution is the use of cell-penetrating proteins or peptides (CPPs). Such peptides enable translocation of the drug of interest coupled to them across the plasma membrane. CPPs are alternatively termed Protein Transduction Domains (TPDs), usually comprise 30 or less (e.g. 5 to 30, or 5 to 20) amino acids, and usually are rich in basic residues, and are derived from naturally occurring CPPs (usually longer than 20 amino acids), or are the result of modelling or design. A non-limiting selection of CPPs includes the TAT peptide (derived from HIV-1 Tat protein), penetratin (derived from Drosophila Antennapedia—Antp), pVEC (derived from murine vascular endothelial cadherin), signal-sequence based peptides or membrane translocating sequences, model amphipathic peptide (MAP), transportan, MPG, polyarginines; more information on these peptides can be found in Torchilin 2008 (Adv Drug Deliv Rev 60:548-558) and references cited therein. The commonly used CPP is the transduction domain of TAT termed TATp, defined by the amino acid sequence YGRKKRRQRRR (SEQ ID No. 80). The MAP peptide is defined by the amino acid sequence KLALKLALKALKAALKLA (SEQ ID No. 81), and the penetratin peptide by RQIKIWFQNRRMKWKK (SEQ ID No. 82). The TAT peptide was e.g. used to shuffle a tau-fragment into neuronal cells (Zhou et al. 2017).

CPPs can be coupled to carriers such as nanoparticles, liposomes, micelles, or generally any hydrophobic particle. Coupling can be by absorption or chemical bonding, such as via a spacer between the CPP and the carrier. To increase target specificity an antibody binding to a target-specific antigen can further be coupled to the carrier (Torchilin 2008, Adv Drug Deliv Rev 60:548-558) CPPs have already been used to deliver payloads as diverse as plasmid DNA, oligonucleotides, siRNA, peptide nucleic acids (PNA), proteins and peptides, small molecules and nanoparticles inside the cell (Stalmans et al. 2013, PloS One 8:e71752).

The present disclosure provides oligonucleotides (oligonucleotides of the present disclosure) comprising an antisense oligomer 10 to 50 (e.g, 12 to 22, or 16 to 21, or 16) contiguous oligonucleotides in length comprising a contiguous sequence at least 10 (e.g., 12 to 22, or 14, 15, 16, 17, 18, 19, or 20) nucleotides in length, which is complementary (e.g., 100% complementary) to a human synaptogyrin-3 target sequence selected from the group consisting of SEQ ID No. 38-54, 69-79, 100-104, 108-109 and 115-121, wherein the antisense oligomer is a gapmer comprising at least one nucleotide variant (e.g., an LNA unit), and wherein the antisense oligomer targets an RNA encoding synaptogyrin-3. In some aspects, the oligonucleotide of the present disclosure further comprises at least one non-nucleotide or non-polynucleotide moiety covalently (e.g., a GalNac moiety) attached to said antisense oligomer directly or via a linker positioned between the contiguous oligomer sequence and the non-nucleotide or non-polynucleotide moiety.

In some aspects, the present disclosure provides oligonucleotides of the present disclosure comprising an antisense oligomer 16 to 22 contiguous oligonucleotides in length comprising a contiguous sequence 16 nucleotides in length, which is 100% complementary to a human synaptogyrin-3 target sequence selected from the group consisting of SEQ ID Nos: 38-54, 69-79, 100-104, 108-109 and 115-121, wherein the antisense oligomer is a gapmer comprising at least one nucleotide variant (e.g., an LNA unit), and wherein the antisense oligomer targets an RNA encoding synaptogyrin-3.

In some aspects, the oligonucleotide of the present disclosure comprises, consists, or consists essentially of an antisense oligomer sequence (antisense to a human synaptogyrin-3 RNA sequence, e.g., mRNA sequence) comprising a sequence selected from the group consisting of SEQ ID No. 7, 16-18, 24 and 55-68.

In some aspects, the oligonucleotide of the present disclosure comprises an antisense oligomer comprising a sequence selected from the group consisting of SEQ ID NO: 7, 16-18, 24 and 55-68, except for 1, 2, 3, 4, 5, 6, 7, 8, 9, 10 nucleobase substitutions. In some aspects, the oligonucleotide of the present disclosure comprises an antisense oligomer comprising a sequence which is at least about 60%, at least about 65%, at least about 70%, at least about 75%, at least about 80%, at least about 85%, at least about 90%, at least about 95%, at least about 96%, at least about 97%, at least about 98%, at least about 99%, or about 100% identical to a sequence selected from the group consisting of SEQ ID NO: 7, 16-18, 24 and 55-68. In some aspects, the oligonucleotide of the present disclosure comprises an antisense oligomer comprising a sequence which is about 60%, about 65%, about 70%, about 75%, about 80%, about 85%, about 90%, about 95%, about 96%, about 97%, about 98%, about 99%, or about 100% identical to a sequence selected from the group consisting of SEQ ID NO: 7, 16-18, 24 and 55-68. In some aspects, the oligonucleotide of the present disclosure comprises an antisense oligomer comprising a sequence that overlaps with 9, 10, 11, 12, 13, 14, 15, or 16 nucleobase subsequence from a sequence selected from the group consisting of SEQ ID NO: 7, 16-18, 24, 55-68.

In some aspects, the oligonucleotide of the present disclosure comprises at least one non-cleavable internucleoside linkage, e.g., a phosphorothioate linkage. In some, aspects all the internucleoside linkages in an oligonucleotide of the present disclosure are non-cleavable, e.g., phosphorothioates linkages. In some aspects, the non-cleavable internucleoside linkages, e.g., a phosphorothioate linkages, are present only in the wing portions of a gapmer, e.g., the last 1, 2 or 3 linkages at the 5′ end or the oligonucleotide, and the last 1, 2 or 3 linkages at the 5′ end or the oligonucleotide. In some aspects, the oligonucleotide of the present disclosure comprises nucleotide analogues. In some aspects, the oligonucleotide of the present disclosure comprises affinity enhancing nucleotide analogues. In some aspects, the nucleotide analogues are sugar modified nucleotides, such as sugar modified nucleotides independently or dependently selected from the group consisting of 2′-O-alkyl-RNA units, 2′-OMe-RNA units, 2′-amino-DNA units, and 2′-fluoro-DNA units.

In some aspects, the oligonucleotide of the present disclosure is a gapmer. In some aspects, the oligonucleotide of the present disclosure a LNA gapmer. In some aspects, the LNA gapmer comprises a wing on each side (5′ and 3′) of 2 to 4 nucleotide analogues, preferably LNA analogues. In some aspects, the oligonucleotide of the present disclosure can optionally comprise a further 1 to 6 nucleotides (e.g., one, two, three, four, five or six nucleotides), which can form or comprise a biocleavable nucleotide region, such as a phosphate nucleotide linker. In some aspects, the biocleavable nucleotide region is formed of a short stretch of nucleotides (e.g. 1, 2, 3, 4, 5 or 6 nucleotides) which are physiologically labile. This can be achieved by using phosphodiester linkages with DNA/RNA nucleosides, or if physiological liability can be maintained, other nucleoside can be used.

In some aspects, the LNA is oxy-LNA, thio-LNA, amino-5 LNA, 5′-methyl-LNA, ENA, cET, cMOE or a combination thereof. In some aspects, the LNA is an stereoisomer in the beta-D-configuration or the alpha-L configuration. In some aspects, the antisense oligomer comprises at least one cET unit. In some aspects, the antisense oligomer comprises 2, 3, 4, 5, 6 or 7 LNA units. In some aspects, every LNA unit in the antisense oligomer is a stereoisomer in the same configuration. In some aspects, every LNA unit in the antisense oligomer is a beta-D-oxy LNA unit or every LNA unit in the antisense oligomer is an alpha-L-oxy-LNA unit. In some aspects, the sequence of the antisense oligomer comprises at least one phosphorothioate, phosphorodithioate, or boranophosphate internucleoside linkage. In some aspects, one or more of the internucleoside linkages comprises a chiral center in the R conformation and/or in the S conformation. In some aspects, the antisense oligomer comprising an LNA can form a duplex with a human synaptogyrin-3 target sequence selected from the group consisting of SEQ ID NOS: 38-54 and 69-79of with increased thermal stability with respect to a corresponding duplex comprising the corresponding antisense oligomer without LNA.

In some aspects, the oligonucleotide of the present disclosure is an antisense oligonucleotide conjugate comprising an antisense oligomer covalently attached to non-nucleotide or non-polynucleotide moiety, which can be attached to the 5′ end, 3′ end, or both. In some aspects, the non-nucleotide or non-polynucleotide moiety is a targeting moiety that is attached to the 5′-end or to the 3′-end of the antisense oligomer. In some aspects, the targeting moiety is linked to the antisense oligomer via a linker. In some aspects, the targeting moiety comprises a carbohydrate conjugate moiety comprising a carbohydrate selected from the group consisting of galactose, lactose, N-acetylgalactosamine (GalNAc), mannose, mannose-6-phosphate, and combinations thereof. In some aspects, the carbohydrate conjugate moiety is not a linear carbohydrate polymer. In some aspects, the carbohydrate conjugate moiety is a carbohydrate group comprising 1, 2, 3, or 4 carbohydrate moieties. In some aspects, the carbohydrate moieties are identical or non-identical. In some aspects, the carbohydrate conjugate moiety comprises at least one asialoglycoprotein receptor targeting conjugate moiety. In some aspects, the asialoglycoprotein receptor targeting conjugate moiety comprises a monovalent, divalent, trivalent, or tetravalent GalNAc cluster. In some aspects, each GalNAc in the GalNAc cluster is attached to a branch point group via a spacer. In some aspects, the branch point group comprises di-lysine. In some aspects, the spacer comprises a PEG spacer. In some aspects, the linker comprises a C6 to C12 amino alkyl group or a biocleavable phosphate nucleotide linker comprising between 1 to 6 nucleotides. In some aspects, the targeting moiety targets the oligonucleotide of the present disclosure to the central nervous system (CNS). In some aspects, the targeting moiety allow the oligonucleotide of the present disclosure to permeate through the BBB.

In some aspects, the oligonucleotide of the present disclosure comprises an antisense oligomer that does not comprise RNA (units), e.g., in some aspects, it can comprise only DNA units. In some aspects, the antisense oligomer comprises DNA and RNA units. In some aspects, the antisense oligomer forms a duplex with a complementary sense oligomer. In some aspects, the sense oligomer and antisense oligomer are connected by a loop, e.g., a loop comprising at least 3 nucleotides. In some aspects, a non-nucleotide or non-polynucleotide moiety can be attached to the 5′ end of the sense oligomer, 3′ end of the sense oligomer, 5′ end of the antisense oligomer, 3′ end of the antisense oligomer, and any combination thereof.

In some aspects, the antisense oligomer is not a double stranded nucleic acid. In some aspects, the antisense oligomer is not a siRNA. In some aspects, the antisense oligomer is not a shRNA. In some aspects, the antisense oligomer is a double stranded nucleic acid. In some aspects, the antisense oligomer is a siRNA. In some aspects, the antisense oligomer of the present disclosure is a shRNA. In some aspects, the antisense oligomer portion of an oligonucleotide of the present disclosure is an antisense oligonucleotide (ASO). In some aspects, the antisense oligomer portion of an oligonucleotide of the present disclosure is multimeric. In some aspects, the antisense oligomer portion of an oligonucleotide of the present disclosure is a multimeric ASO, e.g., it can comprise several concatenated antisense oligomers of the present disclosure. In some aspects, the antisense oligomer portion of an oligonucleotide of the present disclosure comprises 1, 2, 3, 4, 5, 6, 7, 8, 9, 10, or more than 10 concatenated antisense oligomers. In some aspects, the concatenated oligomers are connected via cleavable linkers interposed between each ASO unit in the ASO multimer.

In some aspects, the antisense oligomer portion of an oligonucleotide of the present disclosure can target a target region in the synaptogyrin-3 mRNA selected from the group consisting of SEQ ID No. 38-54, 69-79, 100-104, 108-109 and 115-121. In some aspects, the antisense oligomer portion of an oligonucleotide of the present disclosure targets a target region in the synaptogyrin-3 mRNA of SEQ ID No. 38-54. In some aspects, the antisense oligomer portion of an oligonucleotide of the present disclosure targets a target region in the synaptogyrin-3 mRNA of SEQ ID No. 69-79. In some aspects, the antisense oligomer portion of an oligonucleotide of the present disclosure targets a target region in the synaptogyrin-3 mRNA of SEQ ID No. 100-104. In some aspects, the antisense oligomer portion of an oligonucleotide of the present disclosure targets a target region in the synaptogyrin-3 mRNA of SEQ ID No. 108 or 109. In some aspects, the antisense oligomer portion of an oligonucleotide of the present disclosure targets a target region in the synaptogyrin-3 mRNA of SEQ ID No. 115-121.

In some aspects, the antisense oligomer portion of an oligonucleotide of the present disclosure comprises a complementarity region that is complementary to at least 5, 6, 7, 8, 9, 10, 11, 12, 13, 14, 15, or 16 nucleotides of a target region in the synaptogyrin-3 mRNA selected from the group consisting of SEQ ID No. 38-54, 69-79, 100-104, 108-109 and 115-121. In some aspects, the antisense oligomer portion of an oligonucleotide of the present disclosure comprises a complementarity region that is complementary to at least 5, 6, 7, 8, 9, 10, 11, 12, 13, 14, 15, or 16 nucleotides of a target region in the synaptogyrin-3 mRNA selected from the group consisting of SEQ ID No. 38-54, 69-79, 100-104, 108-109 and 115-121, wherein the complementary region is at the 5′ end of the oligomer. In some aspects, the antisense oligomer portion of an oligonucleotide of the present disclosure comprises a complementarity region that is complementary to at least 5, 6, 7, 8, 9, 10, 11, 12, 13, 14, 15, or 16 nucleotides of a target region in the synaptogyrin-3 mRNA selected from the group consisting of SEQ ID No. 38-54, 69-79, 100-104, 108-109 and 115-121, wherein the complementary region is at the 3′ end of the oligomer. In some aspects, the antisense oligomer portion of an oligonucleotide of the present disclosure comprises a complementarity region that is complementary to at least 5, 6, 7, 8, 9, 10, 11, 12, 13, 14, 15, or 16 nucleotides of a target region in the synaptogyrin-3 mRNA selected from the group consisting of SEQ ID No. 38-54, 69-79, 100-104, 108-109 and 115-121, wherein the complementary region is at the 5′ end of the oligomer, and wherein the oligomer is 12, 13, 14, 15, 16, 17, 18, 19, 20, 21, 22, 23, 24, 25, 26, 27, 28, 29 or 30 nucleotides in length. In some aspects, the antisense oligomer portion of an oligonucleotide of the present disclosure comprises a complementarity region that is complementary to at least 5, 6, 7, 8, 9, 10, 11, 12, 13, 14, 15, or 16 nucleotides of a target region in the synaptogyrin-3 mRNA selected from the group consisting of SEQ ID No. 38-54, 69-79, 100-104, 108-109 and 115-121, wherein the complementary region is at the 3′ end of the oligomer, and wherein the oligomer is 12, 13, 14, 15, 16, 17, 18, 19, 20, 21, 22, 23, 24, 25, 26, 27, 28, 29 or 30 nucleotides in length.

The oligonucleotides of the present disclosure are capable of modulating the expression of the synaptogyrin-3 gene by specifically targeting a targeting region in a synaptogyrin-3 RNA, e.g., an mRNA. In some aspects, the oligonucleotide of the present disclosure is capable of down-regulating expression of the synaptogyrin-3 gene by binding to such target region. Thus, in some aspects, the oligonucleotide of the present disclosure can affect (reduce or inhibit) the expression of synaptogyrin-3, e.g., in a mammalian subject such a human, by binding to a specific target region in a synaptogyrin-3 RNA, e.g., an mRNA. In some aspects, the oligonucleotide of the present disclosure can affect the expression of synaptogyrin-3 in a human cell, by binding to a specific target region in a synaptogyrin-3 RNA, e.g., an mRNA. In some aspects, the RNA is an mRNA, such as pre-mRNA. In some aspects, the RNA is a mature mRNA. The oligomer according to the present disclosure is preferably capable of hybridizing to the target nucleic acid.

In some aspects, the target sequence can extend 1, 2, 3, 4, 5, 6, 7, 8, 9, or 10 nucleotides beyond the 5′ end of a target region of SEQ ID No. 38-54, 69-79, 100-104, 108-109 and 115-121. In some aspects, the target sequence can extend 1, 2, 3, 4, 5, 6, 7, 8, 9, or 10 nucleotides beyond the 3′ end of a target region of SEQ ID No. 38-54, 69-79, 100-104, 108-109 and 115-121. In some aspects, the target sequence can extend 1, 2, 3, 4, 5, 6, 7, 8, 9, or 10 nucleotides beyond the 5′ end and/or 1, 2, 3, 4, 5, 6, 7, 8, 9, or 10 nucleotides beyond the 3′ end of a target region of SEQ ID No. 38-54, 69-79, 100-104, 108-109 and 115-121. In some aspects, the extended target region overlaps with 5, 6, 7, 8, 9, 10, 11, 12, 13, 14, 15, or 16 nucleotides of a target region of SEQ ID No. 38-54, 69-79, 100-104, 108-109 and 115-121.

In some aspects, the target region comprises or consists of a corresponding target sequence region derived from the sequence of mutant or allelic variant of a human synaptogyrin-3 gene encoding the mRNA of SEQ ID No. 1 or SEQ ID No. 3. In other aspects, the target region can be a subsequence present in another mRNA transcript variant encoding human synaptogyrin-3. In some aspects, the target region comprises or consists of a corresponding target sequence region derived from the sequence of a paralog or ortholog of the human synaptogyrin-3 gene encoding the mRNA of SEQ ID No. 1 or SEQ ID No. 3.

In some aspects, the target region is within an exon. In some aspects, the target region is within an intron. In some aspects, the target region comprises the junction between and intron and an exon. In some aspects, the target region is within an intron in the human synaptogyrin-3 pre mRNA.

In some aspects, the oligonucleotides of the present disclosure bind to the target nucleic acid (e.g., SEQ ID No. 38-54, 69-79, 100-104, 108-109 or 115-121) and the effect on synaptogyrin-3 expression and/or activity level is at least about 10% to about 20% reduction in synaptogyrin-3 expression and/or activity level compared to the normal synaptogyrin-3 expression level (e.g., the synaptogyrin-3 expression level of a cell, animal or human treated with saline) and/or normal activity level (e.g. the expression level of a cell, animal or human treated with saline). In some aspects, the reduction in synaptogyrin-3 expression and/or activity is at least about 10%, about least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or about 100% compared to the normal expression and/or activity level. In some aspects, the reduction in expression and/or activity is about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 96%, about 97%, about 98%, about 99%, or about 100% compared to the normal expression and/or activity level.

In some aspects, the synaptogyrin-3 expression and/or activity level after the administration of an oligonucleotide of the present disclosure is less than about 2%, less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, or less than about 80% of the synaptogyrin-3 expression and/or activity level prior to the administration of an oligonucleotide of the present disclosure.

In some aspects, the synaptogyrin-3 expression and/or activity level after the administration of an oligonucleotide of the present disclosure is about 2% to about 5%, about 5% to about 10%, about 10% to about 15%, about 15% to about 20%, about 20%, to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, or about 75% to about 80% of the synaptogyrin-3 expression and/or activity level prior to the administration of an oligonucleotide of the present disclosure.

The present disclosure therefore provides an in vitro or in vivo method of down-regulating or inhibiting the expression of synaptogyrin-3 protein and/or mRNA in a cell which is expressing synaptogyrin-3 protein and/or mRNA, said method comprising administering an oligonucleotide of the present disclosure, e.g., as a pharmaceutical composition of the present disclosure to said cell to down-regulate or inhibit the expression of synaptogyrin-3 protein and/or mRNA in said cell. Suitably the cell is a mammalian cell such as a human cell.

It is to be understood that in some aspects the oligonucleotides of the present disclosure can be multimers comprising, e.g., 2, 3, 4, 5, 6, or more concatenated ASOs disclosed herein, which can optionally be connected by spacers or linkers comprising nucleotide or non-nucleotide units interposed between each ASO in the multimer. Accordingly, in some aspects, the oligonucleotides of the present disclosure can comprise or consist of a contiguous nucleotide sequence of a total of at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 110, at least about 120, at least about 130, at least about 140, at least about 150, at least about 160, at least about 170, at least about 180, at least about 190, or at least about 200 contiguous nucleotides in length.

The present disclosure also provides a pharmaceutical composition comprising an oligonucleotide of the present disclosure (e.g., an unconjugated oligomer or a conjugate) and a pharmaceutically acceptable diluent, carrier, salt, or adjuvant.

The present disclosure provides a method of treating a tauopathic disorder in a subject in need thereof, the method comprising administering comprising administering an effective amount of an oligonucleotide of the present disclosure to the subject. The present disclosure also provides a method of treating or inhibiting progression of a tauopathic disorder or treating or inhibiting a symptom of a tauopathic disorder in a subject in need thereof, the method comprising administering comprising administering an effective amount of an oligonucleotide of the present disclosure to the subject. In some aspects, the tauopathic disorder is selected from the group consisting of Alzheimer's disease, progressive supranuclear palsy (PSP), progressive supranuclear palsy-parkinsonism (PSP-P), Richardson's syndrome, argyrophilic grain disease, corticobasal degeneration Pick's disease, frontotemporal dementia with parkinsonism associated with chromosome 17 (FTDP-17), post-encephalitic parkinsonism, Parkinson's disease complex of Guam, Guadeloupean parkinsonism, Huntington disease, Down's syndrome, dementia pugilistica, familial British dementia, familial Danish dementia, myotonic dystrophy, Hallevorden-Spatz disease, Niemann Pick type C, chronic traumatic encephalopathy, tangle-only dementia, white matter tauopathy with globular glial inclusions, subacute sclerosing panencephalitis, SLC9A6-related mental retardation, non-Guamanian motor neuron disease with neurofibrillary tangles, neurodegeneration with brain iron accumulation, Gerstmann-Straussler-Scheinker disease, frontotemporal lobar degeneration, diffuse neurofibrillary tangles with calcification, chronic traumatic encephalopathy, amyotrophic lateral sclerosis of Guam, amyotrophic lateral sclerosis and parkinsonism-dementia complex, prion protein cerebral amyloid angiopathy, and progressive subcortical gliosis. In some aspects, the symptom of the tauopathic disorder is selected from the group of mild cognitive impairment, dementia, cognitive decline, decline of motor function, oculomotor and bulbar dysfunction, synaptic dysfunction, neurotoxicity, neuronal degeneration, neuronal dysfunction, synapse loss, and amyloid deposition. In some aspects, the synaptic dysfunction is pre-synaptic dysfunction.

The present disclosure provides an in vitro method of reducing expression levels and/or activity of synaptogyrin-3 in a cell comprising administering an effective amount of an oligonucleotide of the present disclosure to the cell. Also provided is a method of reducing expression levels and/or activity of synaptogyrin-3 in a subject in need thereof comprising administering an effective amount of an oligonucleotide of the present disclosure to the subject. Also provided is method of reducing synaptogyrin-3 levels in a subject in need thereof comprising administering to said subject an effective amount of an oligonucleotide of the present disclosure.

The present disclosure also provides a method of manufacturing an oligonucleotide of the present disclosure, the method comprising chemically synthesizing the oligonucleotide of the present disclosure using sequential solid phase oligonucleotide synthesis. The present disclosure provides a method of manufacturing an oligonucleotide of the present disclosure comprising a conjugate moiety, wherein the method comprises covalently attaching the conjugate moiety (e.g., at least one non-nucleotide or non-polynucleotide moiety) covalently to an antisense oligomer disclosed herein. In some aspects, the conjugate moiety (e.g., a non-nucleotide or non-polynucleotide moiety, such as a GalNAc moiety) is attached to an antisense oligomer disclosed herein directly or via a linker positioned between the antisense oligomer sequence and the conjugate moiety. In some aspects, covalently attaching the conjugate moiety (e.g., a non-nucleotide or non-polynucleotide moiety, such as a GalNAc moiety) to the antisense oligomer comprises: (i) chemically synthesizing the antisense oligomer; and, (ii) adding by chemical synthesis or conjugation the conjugate moiety to the antisense oligomer to yield an oligonucleotide conjugate. In some aspects, adding by chemical synthesis or conjugation the conjugate moiety (e.g., a non-nucleotide or non-polynucleotide moiety, such as a GalNAc moiety) to the antisense oligomer to yield an oligonucleotide conjugate comprises: (i) incorporating by chemical synthesis or conjugation at least one conjugate moiety (e.g., a non-nucleotide or non-polynucleotide moiety, such as a GalNAc moiety) to the antisense oligomer; (ii) incorporating by chemical synthesis or conjugation at least one linker to the antisense oligomer or conjugate moiety (e.g., a non-nucleotide or non-polynucleotide moiety, such as a GalNAc moiety); (iii) incorporating by chemical synthesis or conjugation at least one branching point to the antisense oligomer or conjugate moiety (e.g., a non-nucleotide or non-polynucleotide moiety, such as a GalNAc moiety); (iv) incorporating by chemical synthesis or conjugation at least one spacer to the antisense oligomer or conjugate moiety (e.g., a non-nucleotide or non-polynucleotide moiety, such as a GalNAc moiety); or, (v) a combination thereof. In some aspects, (i) at least one linker is interposed between the antisense oligomer and a branching point; (ii) at least one branching point is interposed between a linker and a conjugate moiety (e.g., a non-nucleotide or non-polynucleotide moiety, such as a GalNAc moiety); (iii) at least one, two, or three conjugate moieties (e.g., a non-nucleotide or non-polynucleotide moiety, such as a GalNAc moiety) are attached to a branching point; (iv) at least one polymer spacer (e.g., a PEG spacer) is interposed between a conjugate moiety (e.g., a non-nucleotide or non-polynucleotide moiety, such as a GalNAc moiety) and a branching point; or, (v) any combination thereof.

### Kits and Products of Manufacture

Also provided herein are kits and products of manufacture comprising one or more compositions (e.g., an oligonucleotide of the present disclosure or pharmaceutical compositions comprising an oligonucleotide of the present disclosure) described herein. In some aspects, provided herein is a pharmaceutical pack or kit comprising one or more containers filled with one or more of the ingredients of the pharmaceutical compositions described herein.

In some aspects, the kit or product of manufacture comprises, e.g., a first container comprising a first pharmaceutical composition comprising an oligonucleotide of the present disclosure, a second container containing a solvent, and optionally an instruction for use.

In some aspects, the kit or product of manufacture comprises a container comprising an oligonucleotide of the present disclosure and optionally an instruction for use.

In some aspects, the kit contains a pharmaceutical composition described herein and any prophylactic or therapeutic agent, such as those described herein. In some aspects, the kit further comprises instructions to administer a composition of the present disclosure according to any method disclosed herein. In some aspects, the kit is for use in the treatment of a medical indication disclosed herein. In some aspects, the kit is a diagnostic kit.

All of the references cited above, as well as all references cited herein, are incorporated herein by reference in their entireties.

The sequences of biomolecules (e.g., proteins, genes) disclosed herein and identified by either database accession number or gene name are incorporated by reference. The database accession numbers disclosed herein (e.g, Genbak accession numbers) refer to the database version that in effect on Aug. 10, 2022. The nucleic acid sequences of genes identified by name as well as their official names and alternative names correspond to those in the version of the Genbank database active on Aug. 10, 2022, and are herein incorporated by reference. The amino acid sequences of proteins identified by name or translation products of genes identified by name as well as their official and alternative names correspond to those in the version of the UniProt database active on Aug. 10, 2020, and are herein incorporated by reference.

The following examples are offered by way of illustration and not by way of limitation.

### EXAMPLES

### Example 1. Design and Synthesis of Oligonucleotides

33 oligonucleotides targeting synaptogyrin 3 (Syngr-3) were designed. Five of them targeted intronic regions and 28 targeted the exons. The 33 oligonucleotides comprise siRNAs and ASOs, more particularly LNA (locked nucleic acid)-gapmers. The LNA-gapmers have fully modified phosphorothioate (PS) backbones to avoid enzymatic degradation. The LNA-gapmers were designed based on sequence homology between human and mouse synaptogyrin-3. Mouse and human Syngr-3 full transcripts are 80% identical.

### Example 2. In Vitro Reduction of SYNGR-3 in Neuro2a Mouse Cell Lines Using Oligonucleotides

The oligonucleotides were subsequently tested for their ability to reduce Syngr-3 expression in mouse Neuro-2a neuroblastoma cells. Neuro-2a cells were grown in DMEM supplemented with 10% FBS and plated out in 96-well plates the day before the treatment. The N2a cells were transfected with a single dose of the oligonucleotides at a dose of 50 nM. At 48 h after transfection mRNA was collected and cDNA was prepared. The level of mouse Syngr-3 and that of 2 reference genes (GAPDH and HPRT1) was determined (3 technical repeats) by qPCR using validated primers.

It was found that 15 of the 33 tested oligonucleotides induce a decrease in Syngr-3 levels of more than 50%. The results are shown in Table 1. Interestingly the data show that some regions of the Syngr-3 transcript are suited for oligonucleotide design, while other regions are not accessible for oligonucleotide inhibition.

TABLE 1
Overview of the oligonucleotides tested herein including the location in
the Syngr3 mRNA to which they are complementary. Active means experimental
confirmation of binding to Syngr3 transcript; % KD is % knock-down of
the Syngr3 transcript upon administration of the oligonucleotides.

| | SEQ | location in | | | free | N2A; transfection |
|---|---|---|---|---|---|---|
| compound ID | ID No. | mSYNGR3 | length | transfection | uptake | 50 nM % KD |
| VIB_030_ASO | 5 | 165-181 | 17 | | | 34.86 |
| VIB_018_ASO | 6 | 234-249 | 16 | active | active | 59.51 |
| VIB_017_ASO | 7 | 237-253 | 17 | active | active | 55.98 |
| VIB_041_ASO | 8 | 1128-1143 | 15 | | | 10.82 |
| VIB_037_ASO | 9 | 1144-1159 | 15 | active | | 58.12 |
| VIB_038_ASO | 10 | 1205-1220 | 15 | active | | 86.18 |
| VIB_039_ASO | 11 | 1319-1334 | 15 | active | | 62.94 |
| VIB_040_ASO | 12 | 1861-1876 | 15 | | | 38.40 |
| VIB_024_ASO | 13 | 2271-2287 | 17 | active | | 80.86 |
| VIB_015_ASO | 14 | 2295-2311 | 17 | active | | 44.96 |
| VIB_002_ASO | 15 | 2296-2315 | 20 | | | 19.92 |
| VIB_001_ASO | 16 | 2297-2316 | 20 | active | | 52.70 |
| VIB_006_ASO | 17 | 2297-2316 | 20 | active | | 47.15 |
| VIB_019_ASO | 18 | 2298-2314 | 17 | active | | 63.20 |
| VIB_007_ASO | 19 | 2299-2315 | 17 | | | 37.93 |
| VIB_003_ASO | 20 | 3350-3369 | 20 | | | 27.88 |
| VIB_028_ASO | 21 | 3387-3402 | 16 | active | | 69.35 |
| VIB_005_ASO | 22 | 3466-3485 | 20 | | | 17.14 |
| VIB_011_ASO | 23 | 3470-3486 | 17 | active | | 51.19 |
| VIB_020_ASO | 24 | 3570-3586 | 17 | active | | 64.80 |
| VIB_025_ASO | 25 | 3617-3633 | 17 | active | | 71.75 |
| VIB_012_ASO | 26 | 3707-3720 | 14 | | | 18.70 |
| VIB_016_ASO | 27 | 3707-3723 | 17 | | | 22.36 |
| VIB_004_ASO | 28 | 3707-3726 | 20 | | | 11.77 |
| VIB_014_ASO | 29 | 3710-3726 | 17 | | | 16.94 |
| VIB_013_ASO | 30 | 3713-3726 | 14 | | | 13.66 |
| VIB_026_ASO | 31 | 4550-4566 | 17 | | | 20.45 |
| VIB_027_ASO | 32 | 4550-4566 | 17 | | | −40.78 |
| VIB_034_ASO | 33 | 4554-4570 | 17 | | | 22.16 |
| VIB_033_ASO | 34 | 4561-4577 | 17 | active | | 90.12 |
| VIB_032_ASO | 35 | 4670-4686 | 17 | | | — |
| VIB_031_ASO | 36 | 4683-4699 | 17 | active | | 91.20 |
| VIB_029_ASO | 37 | 4869-4885 | 17 | | | — |

TABLE 3
Sequences and target sequences of active oligonucleotides from Table 1.

| | SEQ | | location | | SEQ |
|---|---|---|---|---|---|
| | ID | | in | Target sequence | ID |
| compound ID | No. | ASO sequence | mSYNGR3 | within SYNGR3 | No. |
| VIB_018_ASO | 6 | CGCAAAGCTCACGGGA | 234-249 | TCCCGTGAGCTTTGCG | 38 |
| VIB_017_ASO | 7 | GCCGCGCAAAGCTCACG | 237-253 | CGTGAGCTTTGCGCGGC | 39 |
| VIB_037_ASO | 9 | ATCATTCCACAATTCT | 1144-1159 | AGAATTGTGGAATGAT | 40 |
| VIB_038_ASO | 10 | GCAAACCACAGTCATT | 1205-1220 | AATGACTGTGGTTTGC | 41 |
| VIB_039_ASO | 11 | GATTCCCGTGAGTAGT | 1319-1334 | ACTACTCACGGGAATC | 42 |
| VIB_015_ASO | 14 | CGTAGCCCTCGTTGACA | 2295-2311 | TGTCAACGAGGGCTACG | 43 |
| VIB_001_ASO | 16 | GTTCACGTAGCCCTCGTTGA | 2297-2316 | TCAACGAGGGCTACGTGAAC | 44 |
| VIB_006_ASO | 17 | GTTCACGTAGCCCTCGTTGA | 2297-2316 | TCAACGAGGGCTACGTGAAC | 44 |
| VIB_019_ASO | 18 | TCACGTAGCCCTCGTTG | 2298-2314 | CAACGAGGGCTACGTGA | 45 |
| VIB_028_ASO | 21 | CGTTGCCACTGATTGG | 3387-3402 | CCAATCAGTGGCAACG | 46 |
| VIB_011 ASO | 23 | CAGCTGAGGATGGAGAA | 3470-3486 | TTCTCCATCCTCAGCTG | 47 |
| VIB_020_ASO | 24 | GGCCTTCACGGTGAGCG | 3570-3586 | CGCTCACCGTGAAGGCC | 48 |
| VIB_025_ASO | 25 | TCTGTGGCAAAGAGAGA | 3617-3633 | TCTCTCTTTGCCACAGA | 49 |
| VIB_033_ASO | 34 | GGATAGAAGATAGACTA | 4561-4577 | TAGTCTATCTTCTATCC | 50 |
| VIB_031_ASO | 36 | GTCTGGAAGAATGAGAC | 4683-4699 | GTCTCATTCTTCCAGAC | 51 |

### Example 3. HIT Validation

To validate the data obtained in Example 2, a free or unassisted uptake experiment was set up using mouse N2a cells and an embryonic mouse Hippocampal cell line (mHippo E-18). mHippo cells are immortalized cell lines from male and female Swiss Webster mouse Hippocampal primary cultures by retroviral transfer of SV40 T-Ag (Cedarlane labs). These cell lines enable accurate in vitro assays for use in the discovery, development and validation of CNS therapies.

The oligonucleotides were incubated together with the cells at a single dose of 1.5 μM for 72 h. Subsequently, mRNA was collected and the level of SYNGR-3 transcripts was determined using qPCR. Interestingly VIB_017 and VIB_018 ASOs were able to reduce the level of Syngr-3 transcript with more than 50%. Importantly, VIB_017 fully matches both the mouse and human sequences while the VIB-018 sequence carries a single mismatch at position 1 to the human sequence.

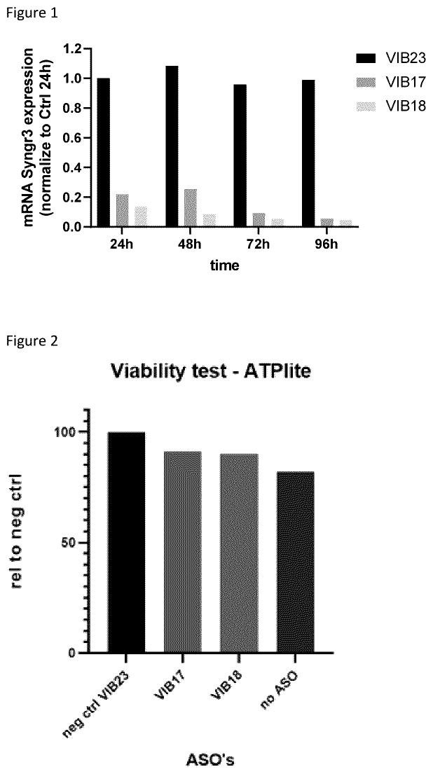

Next, it was tested how fast VIB_017 and VIB_018 can reduce the expression of Syngr-3. A free-uptake experiment using primary mouse neurons was set up in which the ASOs were added at a concentration of 1.5 μM. At different time-points (24 h, 48 h, 72 and 96 h), mRNA was collected and analysed using qPCR. A very strong reduction of Syngr-3 transcripts could already be observed at 24 h, demonstrating that both VIB_017 and VIB_018 administration lead to a fast knock-down of Syngr-3 mRNA levels (FIG. 1).

### Example 4. Target Specificity

During the design of the antisense oligonucleotides, the specificity toward Syngr3 was taken into account. ASO that were found not specific towards Syngr3 in silico were not selected. To experimentally confirm this, the level of Syngr1 was checked in N2A cells. Syngr1 and Syngr3 are 94% identical, however Syngr1 has an extremely long transcript (around 28k bp) compared to Syngr3 (4k bp). Therefore, there is a 3% sequence coverage. Upon treatment of the N2A cells with the Syngr3-specific ASOs, the levels of Syngr1 were unchanged compared to the control treatment. The same could be observed using other targets in primary neuron samples (data not shown).

### Example 5. ASOs Targeting SYNGR-3 do not Affect Neuronal Viability

A free-uptake experiment using primary mouse neurons was set up to test the effect of the ASOs on cell viability. The ASOs were added at a concentration of 1.5 μM and incubated together with the cells for 96 h. As a cell viability readout, we have measured the amount of ATP present in all metabolically active cells. Since ATP concentration decreases rapidly when cell viability is affected, it is a good indicator of cytotoxic and proliferation effects. Compared to the mock treatment or vehicle treated conditions (VIB_023), the on-target ASOs did not induce any toxicity (FIG. 2).

### Example 6. Potency Determination VIB_017 and VIB_018 ASOs

The level of Syngr-3 transcript was measured in primary mouse neurons upon adding Syngr-3 targeting ASOs in a free uptake experiment at different doses. A full dose response analysis was performed using a top concentration of 2 μM with 7 additional steps of 3-fold dilution/step. Analyses were done for VIB_017 and VIB_018. The neurons were incubated together with the ASOs for 96 h.

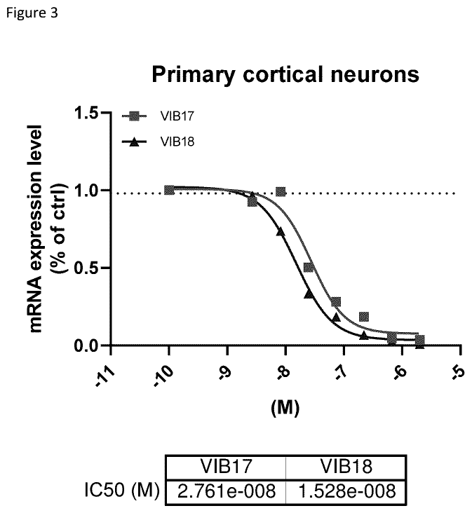

FIG. 3 clearly shows a depletion of the Syngr3 transcript after 96 h in the presence of the active gapmers. Both VIB_017 and VIB_018, show a maximum effect higher than 95%, with a potency ranging 15-30 nM.

### Example 7. Finetuning Targetable Regions within Human SYNGR3

Several of the identified mSyngr-3 regions are conserved in human and most of the oligonucleotides designed above were complementary to regions with the hSyngr-3. Molecule VIB_17 (SEQ ID No. 7) targets 5′-CGTGAGCTTTGCGCGGC-3′ starting at position 237 of the mouse Syngr-3 sequence as depicted in SEQ ID No. 1 and starting at position 494 of the human Syngr-3 sequence as depicted in SEQ ID No. 3. Molecules VIB_001 (SEQ ID No. 16) and VIB_006 (SEQ ID No. 17) target 5′-TCAACGAGGGCTACGTGAAC-3′ starting at position 2297 of the mouse Syngr-3 sequence as depicted in SEQ ID No. 1 and at position 2347 of the human Syngr-3 sequence as depicted in SEQ ID No. 3, while molecule VIB_019 (SEQ ID No. 18) target 5′-CAACGAGGGCTACGTGA-3′ starting at position 2298 of the mouse Syngr-3 sequence as depicted in SEQ ID No. 1 and at position 2346 of the human Syngr-3 sequence as depicted in SEQ ID No. 3. Molecule VIB_020 (SEQ ID No. 24) target 5′-CGCTCACCGTGAAGGCC-3′ starting at position 3570 of the mouse Syngr-3 sequence as depicted in SEQ ID No. 1 and at position 3208 of the human Syngr-3 sequence as depicted in SEQ ID No. 3.

To check whether the regions identified in mSyngr-3 have the same functionality (i.e. suitable regions for designing oligonucleotides that can inhibit the expression of Syngr-3) in human, VIB_017 and VIB_019 were administered to SH-SY5Y cells (human neuroblastoma cell line) at two different concentrations. After an incubation period of 24 h, the transcript levels were determined using the QuantiGene RNA Assay (ThermoFisher) and the results were normalized to a reference gene. VIB_017 reduced the human Syngr-3 expression with 35% at a concentration of 3 nM and with 46.1% at a concentration of 30 nM. Also VIB_019 reduced the expression of hSyngr-3, more particularly up to 50.7% when added to SH-SY5Y cells in a concentration of 30 nM.

Next, an in-dept scanning of the identified regions was done using the human Syngr-3 transcript. Additional 16 bp long oligonucleotides were designed targeting hSyngr-3 fragments upstream and downstream of the identified positions and subsequently tested for their ability to reduce the expression of human Syngr-3 in SH-SY5Y cells (Table 2). These experiments revealed that antisense molecules starting at:
1. a position between 492 and 496 of SEQ ID No. 3 reduced the hSyngr-3 expression with 46.6% to 57.6%. Upstream and downstream of these positions the efficiency of the antisense molecules dropped below 45% inhibition of Syngr-3 expression (Table 2). Indeed, antisense molecules targeting nucleotides 448 to 464 of SEQ ID No. 3 (i.e. R2000252) or targeting nucleotides 447 to 463 of SEQ ID No. 3 (R2000251) only reduced hSyngr-3 expression with 43.6% and 42.8% respectively, while the molecules hybridizing nucleotides 497 to 513 or 498 to 514 of SEQ ID No. 3 (i.e. R2000258 and R2000259) induced only a 40.1% reduction.
2. a position between 2346 and 2350 of SEQ ID No. 3 reduced the hSyngr-3 expression in a range between 48.3 and 50.7%. Upstream and downstream of these positions the efficiency of the antisense molecules dropped below 45% inhibition of Syngr-3 expression (Table 2). An antisense molecule targeting nucleotides 2343-2359 of SEQ ID No. 3 (i.e. R2000444) only reduced hSyngr-3 expression with 31.4%, while the antisense molecules hybridizing nucleotides 2353-2369 (i.e. R2000447) or 2354-2370 (i.e. R2000448) of SEQ ID No. 3 induced a 40.0% and 38.5% reduction in Syngr-3 expression respectively.
3. position between 3188 and 3219 of SEQ ID No. 3 reduced the hSyngr-3 expression in a range between 45.1 and 56.0%. Upstream and downstream of these positions the efficiency of the antisense molecules dropped below 45% inhibition of Syngr-3 expression (Table 2). An antisense molecule (i.e. R2000537) targeting nucleotides 3152-3168 of SEQ ID No. 3 only reduced hSyngr-3 expression with 25.3%. Moving more upstream reduced the efficiency even more. R2000536 targeting nucleotides 3150-3166 of SEQ ID No. 3 reduced Syngr-3 expression with 23.3% and R2000535 hybridizing nucleotides 3147-3163 of SEQ ID No. 3 only lead to a 17.3% reduction in Syngr-3 expression. R2000545 targeting the nucleotides 3221-3237 of SEQ ID No. 3 induced only a 43.2% reduction.

TABLE 2
Sequence of the hSyngr-3 antisense molecules (“ASO sequence”), the regions
within the hSyngr-3 gene to which the molecules bind (“Target sequence within
hSyngr-3”) and the percentage inhibition of hSyngr-3 expression in SH-SY5Y
cells at 3 and 30 nM. The 5′ pos in SEQ ID No. 3 column indicates the 5′ start
of the target sequence in SEQ ID No. 3 to which the ASO binds.

| | 5′ pos in | | SEQ | | SEQ | hSYNGR3 | hSYNGR3 |
|---|---|---|---|---|---|---|---|
| | SEQ ID | | ID | Target sequence | ID | inhib @ 3 | inhib @ 30 |
| ID | No. 3 | ASO sequence | No. | with hSYNGR3 | No. | nM (in %) | nM (in %) |
| R2000249 | 396 | CCTTCTGTCCG | 83 | ACAGGCGGACAG | 96 | 16.2 | 30.3 |
| | | CCTGT | | AAGG | | | |
| R2000250 | 398 | CGCCTTCTGTC | 84 | AGGCGGACAGAA | 97 | 16.7 | 41.4 |
| | | CGCCT | | GGCG | | | |
| R2000251 | 447 | CGAAGGAGGC | 85 | GAGGGCGCCTCCT | 98 | 22.6 | 42.8 |
| | | GCCCTC | | TCG | | | |
| R2000252 | 448 | CCGAAGGAGG | 86 | AGGGCGCCTCCTT | 99 | 30.5 | 43.6 |
| | | CGCCCT | | CGG | | | |
| R2000253 | 492 | GCGCAAAGCT | 55 | CCCGTGAGCTTTG | 100 | 27.9 | 52.1 |
| | | CACGGG | | CGC | | | |
| R2000254 | 493 | CGCGCAAAGC | 56 | CCGTGAGCTTTGC | 101 | 37.5 | 57.6 |
| | | TCACGG | | GCG | | | |
| R2000255 | 494 | CCGCGCAAAG | 57 | CGTGAGCTTTGCG | 102 | 35.0 | 46.1 |
| | | CTCACG | | CGG | | | |
| R2000256 | 495 | GCCGCGCAAA | 58 | GTGAGCTTTGCGC | 103 | 35.4 | 47.7 |
| | | GCTCAC | | GGC | | | |
| R2000257 | 496 | CGCCGCGCAA | 59 | TGAGCTTTGCGCG | 104 | 31.8 | 46.6 |
| | | AGCTCA | | GCG | | | |
| R2000258 | 497 | CCGCCGCGCA | 87 | GAGCTTTGCGCG | 105 | 27.1 | 40.1 |
| | | AAGCTC | | GCGG | | | |
| R2000259 | 498 | GCCGCCGCGC | 88 | AGCTTTGCGCGGC | 106 | 24.3 | 40.1 |
| | | AAAGCT | | GGC | | | |
| R2000444 | 2343 | TAGCCCTCGTT | 89 | TCGTCAACGAGG | 107 | -1.6 | 31.4 |
| | | GACGA | | GCTA | | | |
| R2000445 | 2346 | ACGTAGCCCT | 60 | TCAACGAGGGCTA | 108 | 11.4 | 50.7 |
| | | CGTTGA | | CGT | | | |
| R2000446 | 2350 | GTTCACGTAG | 61 | CGAGGGCTACGT | 109 | 24.7 | 48.3 |
| | | CCCTCG | | GAAC | | | |
| R2000447 | 2353 | GGTGTTCACG | 90 | GGGCTACGTGAA | 110 | 6.8 | 40.0 |
| | | TAGCCC | | CACC | | | |
| R2000448 | 2354 | CGGTGTTCAC | 91 | GGCTACGTGAACA | 111 | 14.3 | 38.5 |
| | | GTAGCC | | CCG | | | |
| R2000535 | 3147 | GGATCAGCCG | 92 | GGCGCTCGGCTG | 112 | −0.2 | 17.3 |
| | | AGCGCC | | ATCC | | | |
| R2000536 | 3150 | CCGGGATCAG | 93 | GCTCGGCTGATCC | 113 | 11.5 | 23.3 |
| | | CCGAGC | | CGG | | | |
| R2000537 | 3152 | AGCCGGGATC | 94 | TCGGCTGATCCCG | 114 | 6.7 | 25.3 |
| | | AGCCGA | | GCT | | | |
| R2000538 | 3188 | TGAGCGCCAC | 62 | GCGCAGGTGGCG | 115 | 7.3 | 45.1 |
| | | CTGCGC | | CTCA | | | |
| R2000539 | 3192 | ACGGTGAGCG | 63 | AGGTGGCGCTCAC | 116 | 18.1 | 45.3 |
| | | CCACCT | | CGT | | | |
| R2000540 | 3194 | TCACGGTGAG | 64 | GTGGCGCTCACCG | 117 | 39.7 | 50.3 |
| | | CGCCAC | | TGA | | | |
| R2000541 | 3195 | TTCACGGTGA | 65 | TGGCGCTCACCGT | 118 | 41.4 | 48.5 |
| | | GCGCCA | | GAA | | | |
| R2000542 | 3196 | CTTCACGGTG | 66 | GGCGCTCACCGTG | 119 | 42.6 | 56.0 |
| | | AGCGCC | | AAG | | | |
| R2000543 | 3214 | GCGGAACCGC | 67 | CCTGCAGCGGTTC | 120 | 33.3 | 50.2 |
| | | TGCAGG | | CGC | | | |
| R2000544 | 3219 | CCCAGGCGGA | 68 | AGCGGTTCCGCCT | 121 | 27.6 | 52.2 |
| | | ACCGCT | | GGG | | | |
| R2000545 | 3221 | TGCCCAGGCG | 95 | CGGTTCCGCCTGG | 122 | 28.7 | 43.2 |
| | | GAACCG | | GCA | | | |

To conclude, it was shown that particular regions with the mouse and human Syngr-3 transcript are more or less suitable for designing antisense oligonucleotides. Given that a heterozygous Syngr-3 knock-out can rescue Tau pathology in mice (McInnes et al 2018 Neuron 97: 823-835), it was anticipated that any Syngr-3 antisense molecule reducing the expression of hSyngr-3 with at least 45% can potentially be part of a tauopathology treatment. Hence, the regions within the hSyngr-3 transcript between position 470 and 512, between position 2344 and 2368 and between position 3170 and 3236 of SEQ ID No. 3 are of particular interest for designing antisense molecules for a treatment of tauopathies.

### Example 8. Oligonucleotides Reducing Human Syngr-3 Transcript to Therapeutic Levels

Some of the oligonucleotides of Example 7 were further tested in additional cells. N2A cells were transfected with selected oligonucleotides for 48 h in a dose-response curve. In particular, R2000255 is highly active towards Syngr3 (71.8%) with an IC50 of ^{˜}10 nM. No toxic effects were observed. Additionally, this compound was tested in human iPSC-derived neurons, under gymnotic uptake, and was reducing the expression levels of Syngr3 to ^{˜}50% (5 μM). Similarly, R2000540 inhibits Syngr3 expression by 50% in N2A cells with a calculated IC50 of 5.9 nM, under transfection conditions. In human iPSC-derived neurons, R2000540 inhibits the expression of the target by 85%.

### Experimental Procedures

### Cell Culture Neuro-2A

Neuro-2a cells were grown in DMEM supplemented with 10% FBS and plated out in 96-well plates the day before the treatment. On the day of the treatment, compounds were added to the cells and incubated for the duration of the treatment. Medium was kept constant and cells were undisturbed throughout the study.

### Mouse Primary Neurons

Neurons were cultured from E18 to E19 WT C57BL/6J mice embryos. Briefly, dissected cortices were incubated with trypsin (0.25% [vol/vol], 15 min, 37° C.) in HBSS supplemented with 10 mM HEPES. After trypsin incubation, cortices were washed with MEM (Thermo Fisher Scientific, Waltham, MA) supplemented with 10% (v/v) horse serum (Thermo Fisher Scientific, Waltham, MA) and 0.6% (wt/vol) glucose (MEM-horse serum medium) 3 times. The cells were mechanically dissociated by repeatedly pipetting the tissue up and down in a flame-polished Pasteur pipette and then plated on poly-D-lysine (Millipore, Burlington, MA) coated 96-well plates. To prevent overgrowth of glia, neuron cultures were treated with 10 μM 5-Fluoro-2′-deoxyuridine (Sigma-Aldrich, St. Louis, MO,) after 3 d. Cultures were maintained in a humidified incubator of 5% (vol/vol) CO₂/95% (vol/vol) air at 37° C., feeding the cells at day 7.

### Compound Incubation

ASOs were suspended in H₂O at a stock concentration of 50 μM, aliquot and kept at −80 C until further use. For transfection studies, FuGENE has been used. Shortly, FuGENE and each compound (final concentration 50 nM) are added together for 15 min at room temperature, and later added to the culture. For free up-take conditions, ASOs were diluted in OPTI-MEM before adding to the cells.

RNA Extraction and qPCR

For RNA extraction and qPCR analysis, ‘cells-to CT kit’ (ThermoFisher) was used following manufacture instructions. Shortly, cells are lysed for five minutes followed by the addition of a STOP solution added for 2 min. This lysate is then used for preparation of cDNA. For the determination of Syngr3 expression levels, a qPCR assay was done using two reference genes for normalizing the data (GAPDH and HPRT1). All primers used have been validated beforehand. qPCR was run in a QuantStudio 5 RealTime PCR machine and data was analyzed using a dedicated software (GenEX).

### ASOs Testing—Transfection in SH-SY5Y, Dual Dose

SH-SY5Y cells were seeded at a density of 20.000 cells/well on collagen-coated 96-well tissue culture plates, followed by transfection of cells using Dharmafect-4 (0.5l/well). The two final ASOs concentrations tested were 30 nM and 3 nM. Cells were incubated for 24 h at 37° C./5% CO₂ in a humidified incubator, followed by cell lysis and bDNA analysis to monitor on-target mRNA expression levels relative to hsGAPDH mRNA levels. The mean ratio of on-target hsSYNGR3/hsGAPDH was artificially set to 100% and used to normalize all other samples. All data were generated in quadruplicates.

ASOs Testing—Gymnotic Uptake in Cortical Neurons Derived from Human iPSC

Cortical neuronal precursor cells were seeded at a density of 35.000 cell/well on a PDL/mlaminin coated 96w plates. After 21 days of culture in Terminal Differentiation Medium cells were treated with ASOs.

During the 21 days of differentiation, medium was partially replaced twice a week. ASOs were added to the cells at a 1.5× concentration, to a final concentration of 1 and 5 μM. Cells were incubated with ASO's for 96 h at 37° C./5% C02 in a humidified incubator, followed by cell lysis and qPCR analysis to monitor on-target mRNA expression levels relative to hGAPDH and hHPRT1. The mean ratio of on-target hsSYNGR3/(hsGAPDH+hHPRT1) was artificially set to 100% and used to normalize all other samples.

1. An oligonucleotide of 10 to 50 nucleotides in length, wherein the oligonucleotide comprises a contiguous nucleotide sequence of at least 10 nucleotides in length that is at least 90% complementary to a region of equal length of Synaptogyrin-3 as set forth in SEQ ID No. 1 or SEQ ID No. 3, and wherein the region is comprised within a sequence set forth in SEQ ID No. 38-54 or SEQ ID No. 69-79.

2. The oligonucleotide of claim 1, wherein the contiguous nucleotide sequence is at least 15 nucleotides in length.

3. The oligonucleotide of claim 1, wherein the region is selected from SEQ ID Nos. 53-54, SEQ ID Nos. 69-79, SEQ ID Nos. 100-104, SEQ ID Nos. 108-109, and SEQ ID Nos. 115-121.

4. The oligonucleotide of claim 1, wherein the contiguous nucleotide sequence is 100% complementary to:
a region of equal length of Synaptogyrin-3 as set forth in SEQ ID No. 1 or SEQ ID No. 3, wherein the region is comprised within a sequence set forth in SEQ ID No. 38-54 or SEQ ID No. 69-79; and/or
a region selected from SEQ ID Nos. 53-54, SEQ ID Nos. 69-79, SEQ ID Nos. 100-104, SEQ ID Nos. 108-109, and SEQ ID Nos. 115-121.

5. The oligonucleotide of claim 1, wherein the oligonucleotide comprises one or more internucleoside linkages and/or one or more 2′ sugar modified nucleosides.

6. The oligonucleotide of claim 5, wherein the internucleoside linkage is a phosphorothioate internucleoside linkage and/or the 2′ sugar modified nucleoside is selected from the group consisting of 2′-O-methyl-, 2′-O-methoxyethyl-, 2′-O-alkyl-, 2′-alkoxy, 2′-amino-, 2′-fluoro-, and LNA nucleosides.

7. The oligonucleotide of claim 1, wherein the oligonucleotide is a single stranded antisense oligonucleotide, an siRNA, a shRNA, a CRISPR gRNA, or forms the guide strand of an siRNA or shRNA complex.

8. The oligonucleotide of claim 1, wherein the oligonucleotide comprises a gapmer of the formula 5′-F-G-F′-3′, where F and F′ are regions and independently comprise between 1 and 8 nucleosides, of which 1 to 5 independently are 2′ sugar modified nucleosides and define the 5′ and 3′ ends of the F and F′ regions, and wherein G is a region between 5 and 18 nucleosides which recruits RNaseH.

9. The oligonucleotide of claim 8, wherein the internucleoside linkages between one or more nucleosides of region F and/or F′, and/or between F and G, and/or between F′ and G regions are phosphorothioate internucleoside linkages.

10. The oligonucleotide of claim 1, wherein the oligonucleotide is comprised in a pharmaceutical composition.

11. (canceled)

12. A method of treating, treating a symptom of, or inhibiting the progression of a tauopathic disorder in a subject the method comprising: administering to the subject the oligonucleotide of claim 1.

13. The method according to claim 12, wherein the tauopathic disorder is selected from the group consisting of Alzheimer's disease, progressive supranuclear palsy (PSP), progressive supranuclear palsy-parkinsonism (PSP-P), Richardson's syndrome, argyrophilic grain disease, corticobasal degeneration Pick's disease, frontotemporal dementia with parkinsonism associated with chromosome 17 (FTDP-17), post-encephalitic parkinsonism, Parkinson's disease complex of Guam, Guadeloupean parkinsonism, Huntington disease, Down's syndrome, dementia pugilistica, familial British dementia, familial Danish dementia, myotonic dystrophy, Hallevorden-Spatz disease, Niemann Pick type C, chronic traumatic encephalopathy, tangle-only dementia, white matter tauopathy with globular glial inclusions, subacute sclerosing panencephalitis, SLC9A6-related mental retardation, non-Guamanian motor neuron disease with neurofibrillary tangles, neurodegeneration with brain iron accumulation, Gerstmann-Straussler-Scheinker disease, frontotemporal lobar degeneration, diffuse neurofibrillary tangles with calcification, chronic traumatic encephalopathy, amyotrophic lateral sclerosis of Guam, amyotrophic lateral sclerosis and parkinsonism-dementia complex, prion protein cerebral amyloid angiopathy, and progressive subcortical gliosis.

14. The method according to claim 12 wherein the symptom of the tauopathic disorder is selected from the group of mild cognitive impairment, dementia, cognitive decline, decline of motor function, oculomotor and bulbar dysfunction, synaptic dysfunction, neurotoxicity, neuronal degeneration, neuronal dysfunction, synapse loss, and amyloid deposition.

15. The method according to claim 14 wherein the synaptic dysfunction is pre-synaptic dysfunction.
